(19) United States
(12) Patent Application Publication
(10) Pub. No.: US 2004/0132132 A1
(43) Pub. Date: Jul. 8, 2004

(54) Method for identifying biologically active structures of microbial pathogens

(76) Inventors: Ugur Sahin, Mainz (DE); Ozlem Tuereci, Mainz (DE); Burkhard Ludewig, Mainz (DE)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/468,591

(22) PCT Filed: Feb. 22, 2002
(86) PCT No.: PCT/EP02/01909

(30) Foreign Application Priority Data
Feb. 22, 2001 (DE) 10108626.1

(51) Int. Cl. C12Q 1/68; C12P 21/06

(52) U.S. Cl. 435/69.1

(57) ABSTRACT

The present invention concerns a method for identifying biologically active structures which are coded by the genome of microbial pathogens, using genomic pathogen nucleic acids.

The invention described below concerns a procedure for identifying biologically active structures which are coded by the genome of microbial pathogens, on the basis of genomic pathogenic nucleic acids.

### BACKGROUND OF THE INVENTION

A requirement for the development of molecularly defined serodiagnostic agents and vaccines is the molecular knowledge and availability of the antigens of the pathogenic agent (the microbial immunome) recognized by the immune system of an infected host. Serodiagnosis of infectious diseases is based on the detection of antibodies circulating in the blood, which are directed specifically against immunogenic components (antigens) of the pathogen and thus indicate an existing or recent infection. Knowledge of these antigens makes it possible to produce antigens through recombination as molecularly defined vaccines. These vaccines can give an organism protection against an infection caused by the pathogen in question (prophylactic immunization), but also, in the case of persistent and chronic pathogens, serve to eliminate them (therapeutic immunization). The significance of such antigens for both a specific diagnosis and a specific therapy has resulted in considerable interest in the identification of these structures.

Although most relevant antigens have been identified molecularly in the case of low-complex infection pathogens with a known genome (e.g. simple viruses), it is still not clear which antigens are immunologically relevant in the case of more complex infection pathogens (e.g. bacteria). The reason for this is that the complex genomes of these pathogens contain a large number of genes (1000 to over 4000), which makes a quick identification of the relevant antigens difficult. Even in the case of pathogens whose genomes are entirely known, one must assume that not all nucleotide regions have been attributed to segments that are actually coding for proteins—and are thus potentially antigen-producing segments.

A number of technologies that have been developed over the past years for identifying antigens, attempt to deal with this complexity. Most of these methods come from the field of “proteomics” technologies, the name for high-throughput protein analysis technologies.

One of these high-throughput technologies includes the use of 2-D gels (e.g. Liu B, Marks J D. (2000), Anal. Biochem. 286,1191-28). Since large numbers of pathogens are required in this method, these are first multiplied under culture conditions. Extracts from lysed pathogens are then produced and the proteins contained in them separated by gel electrophoresis. Protein complexes identified by means of immune serums (patients' serums, serums from immunized animals) can be analyzed through isolation and microsequencing. This method has a number of limitations and disadvantages, since large amounts of pathogenic material are needed for the analyses. Analysis is not possible directly from the primary lesion, but only after often very time-consuming culturing (e.g. in the case of mycobacteria). Some pathogens cannot be cultured in a simple manner; for unknown pathogens, culturing conditions are often not defined. Dead pathogens are generally excluded from this enrichment method.

Another disadvantage of the 2-D gel technology is that the gene expression status of a pathogen in a cell culture is clearly different from that in vivo. Many pathogen gene products are engaged only when a pathogen invades the host organism. For this reason, for the analysis only such proteins are available that are expressed in the infection pathogen at the time of culturing. This rules out a number of proteins that are expressed in the host in detectable amounts only under infection conditions. However, those very proteins can be relevant for diagnostic serology, which must be able to distinguish between a clinically irrelevant colonization and an invasive infection.

Also, antigens are identified as proteins by means of 2-D gels. The nucleotide sequence that is the basis for many subsequent analyses must still be determined.

As an alternative to the 2-D gel method, it is possible in pathogens whose entire nucleotide sequence is known, to introduce all putative genes into expression cassettes, express them in recombination and examine them for immunogenity or antigenity (Pizza et al. (2000), Science 287(5459):1816-20). The genes expressed through recombination are screened, for example, in a parallelized dot blot method with immunoserums.

The disadvantage of this method is that only proteins which are known to be expressed in the pathogen of interest can be analyzed. Pathogens with an unknown nucleotide sequence cannot be detected with this analysis.

Since the aforementioned analytical technologies require a great amount of material, time, staff and costs, they are reserved for only a few large centres.

The immunoscreening of genomic expression banks (e.g. Pereboeva et al. (2000), J. Med. Virol. 60: 144-151) is an efficient and potentially effective alternative to the “proteomics” approaches. However, for this purpose it is also necessary to enrich infection pathogens under defined culturing conditions. The genome of the pathogens is subsequently isolated, chopped up into fragments enzymatically or mechanically, and finally cloned in expression vectors. The expressed fragments can then be examined to determine whether they are recognized by serums from infected organisms. The advantage of this method is that it can provide cost-effective and rapid identification of antigens. However, for this method as well, it is essential, by reason of the large amount of the required pathogen nucleic acids, to multiply the pathogens through in vitro culturing and then re-purify them. Consequently, the method has hitherto been limited to pathogens whose culturing and purification modalities are known and established.

One problem is to identify infection pathogens which have so far not been characterized or only insufficiently characterized. The present invention deals in particular with the following [SH1] challenges:

1) Inflammatory diseases whose cause, based on epidemiology and their clinical course, is likely to be an infection whose pathogens cannot be defined and/or only insufficiently characterized by means of known methods. This includes diseases such as multiple sclerosis, Kawasaki's disease, sarcoidosis, diabetes mellitus, morbus Whipple, pityriasis rosea, etc. It would be desirable to have a method for these diseases that allows a systematic analysis for determining unknown infection pathogens from primary patient material such as lymph node biopsies.

2) Newly emerging infectious diseases. This includes infectious diseases caused by hitherto unknown or not well-characterized pathogens (e.g. HIV in the 80s) und which, for example by reason of a change in epidemiology, are suddenly the focus of clinical interest. From a medical and socioeconomical point of view, rapid pathogen identification, the development of corresponding diagnostics and, possibly, the production of vaccines are essential. Since, in general, establishing culturing conditions for non-characterized pathogens may take up to several years, it is highly desirable, in this case as well, to identify pathogens and pathogen antigens directly from the infected tissue; however, this cannot be done by using the known methods.

There is therefore a great demand for a method allowing the direct use of primary material for pathogen identification without the use of pathogen cultures. In addition, this method should allow the efficient discovery of hitherto unknown pathogens in primary material.

### ABSTRACT OF THE INVENTION

One purpose of the present invention was therefore to develop a method allowing identification of pathogen nucleic acids directly from a limited amount (e.g. 50 mm³) of infected patient material.

The present invention describes a method for the systematic identification of known as well as unknown nucleic acid coded pathogens and their antigens, using the immunological response triggered by them in the host organism.

The subject matter of the present invention is therefore a method for identifying biologically active structures that are coded by the genome of microbial pathogens, using genomic pathogen nucleic acids, the method including the following steps: extraction of genomic pathogen nucleic acids from samples containing pathogens, sequence-independent amplification of genomic pathogen nucleic acids, expression of amplified pathogen nucleic acids and screening and identification of the biologically active structure.

The method according to the invention has the decisive advantage that a comprehensive identification of pathogen antigens recognized by the host organism (microbial immunone) is possible even for very small amounts of pathogens.

The method according to the invention is characterized in that minimal initial amounts of as little as 1 pg of pathogen nucleic acid are sufficient to perform an effective analysis. In a preferred realization one uses 10-20 pg, more specifically 1-10 pg of pathogen nucleic acid.

The high sensibility of the method makes it possible, on the one hand, to analyse pathogens from primary isolates without having to enrich these pathogens by in vitro culturing beforehand. This way it is possible to examine pathogens which can only with difficulty be cultured with known methods (e.g. mycobacterium tuberculosis etc.) or cannot be cultured at all (e.g. mycobacterium leprae or non-vital germs).

On the other hand, when in vitro enrichment is no longer necessary, one can avoid a contamination of the germ population (e.g. by excessive growth of relevant pathogenic germs in mixed infections) caused by in vitro culturing.

In addition, the high sensitivity of the method makes it possible to use a broad spectrum of source materials for pathogen isolation. The term of “sample”, in this context, refers to different biological materials such as cells, tissue, body fluids. In a preferred realization, blood, tissue, cultured cells, serum, secretions from lesions (pustules, scabs, etc.) and other body fluids such as urine, saliva, liquor, joint fluids, gall and eye gland fluids are preferably used samples.

### DETAILED DESCRIPTION OF THE INVENTION

Obtaining genomic pathogen nucleic acids from samples containing pathogens

In the first step of the method according to the invention, genomic pathogen nucleic acids are obtained from samples containing pathogens.

The term “microbial pathogen” used here comprises viral and bacterial pathogens. Pathogens are present in host cells or in cell combinations with host cells, and, with the exception of pathogens circulating in the serum, must be made accessible. In a preferred embodiment of the invention, the pathogens are present intracellularly or extracellularly.

Intracellular pathogens can be released by cytolysis (e.g. mechanically or by means of detergents, with eukaryotic cell membranes, for example, by means of SDS). Preferred SDS concentrations are for gram-positive bacteria >0.05% to 1%, and for gram-negative bacteria 0.05% to 0.1%. The person skilled in the art can easily determine the suitable concentrations for other detergents by using that concentration at which the envelope, e.g. the wall of the gram-positive or gram-negative bacterium is still intact, but at which the eukaryotic cell wall is already dissolved.

Extracellular pathogens can be separated from host cells, for example, because of their perceptibly smaller particle size (20 nm-1 μM) (e.g. by sedimentation/centrifugation and/or filtration). In a particularly preferred embodiment of the invention, the pathogen is not infectious and/or not vital. The high sensitivity of the method makes it possible for recourse to be made even to non-infectious pathogens and/or to residual non-vital pathogens remaining after the course of a florid infection.

The first step of the method according to the invention is further described hereinafter, namely, the obtaining of genomic pathogen nucleic acids from samples containing pathogens.

The obtaining of genomic pathogen nucleic acids from samples containing pathogens is characterized in a preferred embodiment of the invention by the following steps: release of pathogen particles from samples containing pathogens, followed by the elimination and/or reduction of contaminating host nucleic acids and subsequent extraction of the genomic pathogen nucleic acid from released pathogen particles.

According to one embodiment of the invention, the release of pathogen particles is effected by cytolysis, sedimentation, centrifugation, and/or filtration.

According to a preferred embodiment, the elimination of the contaminating host nucleic acids is effected by an RNase and/DNase digestion process being carried out prior to the extraction of the pathogen nucleic acids.

This is of particular advantage if the pathogen is a virus protected by capsid proteins. Because the capsid envelopes of viruses provide protection against nucleases, the pathogens are accordingly protected against the activity of extracellular RNases and DNases. A further embodiment of the invention therefore comprises the step of eliminating and/or reducing contaminating host nucleic acids by RNase and/or DNase digestion, in particular for viral pathogens (such as vaccinia virus). The DNase treatment for the purification of virus particles is known to the person skilled in the art and can be carried out as described, for example, by Dahl R., Kates J R., Virology 1970; 42(2): 453-62, Gutteridge W E., Cover B., Trans R Soc Trop Med Hyg 1973; 67(2):254), Keel J A. Finnerty W R, Feeley J C., Ann Intern Med 1979 April; 90(4):652-5 or Rotten S. in Methods in Mycoplasmology, Vol. 1, Academic Press 1983.

Another possibility of enriching pathogens such as gram-positive or gram-negative bacteria from tissue is, as applied in the method according to the invention, to use a differential lysis (also designated hereinafter as sequential lysis) of infected tissue with detergents such as, for example, SDS, which dissolve the lipid membranes. In this situation, use is made of the fact that the cell membranes of eukaryotic host cells react with very much greater sensitivity to low concentrations of SDS, and are dissolved, while bacteria walls are more resistant and their corpuscular integrity is maintained.

After enrichment, the pathogen nucleic acids are separated from the corpuscular components of the pathogen and released from the pathogen. This can be carried out by the person skilled in the art by known standard techniques, whereby in a preferred embodiment of the invention the separation takes place by means of proteinase K digestion, denaturation, heat and/or ultrasound treatment, enzymatically by means of lysozyme treatment, or organic extraction.

The purification of bacteria/viruses by detergents such as SDS can be carried out as described by Takumi K., Kinouchi T., Kawata T., Microbiol Immunol 1980; 24(6):469-77, Kramer V C., Calabrese D M., Nickerson K W., Environ Microbiol 1980 40(5):973-6, Rudoi N M., Zelenskaia A B., Erkovskaia, Lab Delo 1975;(8): 487-9 or Keel J A., Finnerty W R., Feeley J C., Ann Intern Med 1979 April; 90(4):652-5, see also U.S. Pat. No. 4,283,490.

The invention resolves the problem that in an infected tissue/organ only a part of the tissue/cells is infected with the pathogen.

The present invention is also suitable for providing evidence of pathogens in cases such as, for example, mycobacterioses, in which only few or isolated pathogen particles exist in the infected tissues.

The present invention has the advantage that in cases of an infection with only a small total quantity of pathogen nucleic acids per tissue unit (e.g. 50 mm³), the detection of the pathoaen is possible. As an example for numbers of DNA-coded pathogens, it is possible to calculate that approximately 10⁶ viruses with an average genome size of 10,000 bases or 10⁴ bacteria with an average genome size of 1,000,000 bases contain just 10 pg of pathogen nucleic acids. In addition, the genome of most infection pathogens is perceptibly smaller than the human genome (up to a factor of 10⁶ for viruses, and up to a factor of 10⁴ for bacteria). As a result, the proportion of the total volume of pathogen nucleic acids in relation to the total volume of host nucleic acids is diluted, depending on the number of copies and the size of the pathogen genome, by a multiple of powers to the tenth (ratio of host nucleic acids to pathogen nucleic acid 10⁴ to 10⁹).

Sequence-independent Amplification of Genomic Pathogen Nucleic Acids

The method according to the invention further comprises the sequence-independent amplification of genomic pathogen nucleic acids by means of polymerase chain reaction (PCR), which serves to increase the source material.

In this context, PCR primers are used in random sequences (random oligonucleotides), with the result that not only specific gene ranges can be amplified in a representative manner, but, as far as possible, the entire genome of the pathogen (non-selective nucleic acid amplification with degenerated oligonucleotides). These primers naturally also bond with the host DNA, but, as described earlier, they were reduced or eliminated by previous RNase and/or DNase digestion or by selective cell lysis.

However, elimination or reduction of host DNA by prior RNase and/or DNase digestion or by selective cytolysis respectively is not absolutely necessary in the method according to the invention. With a high concentration of the pathogen DNA or RNA, such as occurs, for example, in pustules or virus blisters, the elimination or reduction of host DNA is not required.

The term “genomic pathogen nucleic acid” used here comprises both genomic DNA as well as RNA.

In a preferred embodiment of the invention, the genomic pathcgen nucleic acid is DNA, and its sequence-independent amplification is effected by Klenow's reaction with adaptor oligonucleotides with degenerated 3′ end and subsequent PCR with oligonucleotides, which correspond to the adaptor sequence.

In a further preferred embodiment, the genomic pathogen nucleic acid is RNA, and its amplification is effected by reverse transcription with degenerated oligonucleotides and subsequent amplification by PCR.

If it is not known whether the pathogen is coded by RNA or DNA, both reactions are implemented in separate reaction vessels and separately amplified. The amplicons represent in both cases genomic nucleic acid fragments. By using degenerated oligonucleotides, a random amplification (shotgun technique) of the entire genome is made possible.

The strength of the method according to the invention lies in its high sensitivity and efficiency (initial amounts of only a few picograms are sufficient), while, at the same time, the representation of all sectors of the whole genome is well maintained. The good representation of the microbial gene segments in the libraries generated by the method according to the invention is achieved by variations in the two-step PCR, such as, for example, changes in the salt concentration. The high efficiency of the method, with the extensive maintenance of representation, therefore makes the preparation of a primary culture for the multiplication of the pathogen unnecessary, with all the limitations that the multiplication involves. In the case of unknown pathogens, culture conditions are not defined and would have to be approximated by the trial-and-error method. Moreover, a series of known pathogens is difficult to cultivate. With mixed infections, primary cultures are capable of specifically diluting the relevant pathogen population by means of overgrowth phenomena. Dead pathogens would not be detected at all. These disadvantages of other techniques are circumvented by the method according to the invention.

In a preferred embodiment, the sequence non-specific amplification of pathogen nucleic acids is carried out in two sequential PCR steps applied one after the other, of35-40 cycles in each case.

To do this, 1/20 to 1/50 of the volume of the first PCR is used after the first amplification for the re-amplification under varying conditions (e.g. variation of the MgCl concentration, the buffer conditions, or the polymerases). As a result of the re-amplification in a second PCR, as represented in Example 3A, a higher sensitivity of the method is guaranteed. The variation of the re-amplification conditions (see Example 6, FIG. 6) makes possible an especially good representation of different segments of the pathogen genome, and therefore a comprehensive analysis of the pathogen.

Expression of Amplified Pathogen Nucleic Acids

The amplification of the genomic pathogen nucleic acids is followed by their expression. To do this, the pathogen nucleic acids are cloned in order to produce a genomic expression bank of the pathogen into suitable expression vectors.

In one embodiment of the invention, the expression vectors are selected from the group of viral, eukaryotic, or prokaryotic vectors. Within the framework of the invention, all systems can be used which permit an expression of recombined proteins.

Following the introduction of pathogen nucleic acids into the vectors, the vectors are preferably packaged in lambda phages.

In a preferred embodiment of the invention, the expression of the pathogen nucleic acids is guaranteed by the introduction of the pathogen nucleic acids into lambda phage vectors (e.g. lambda ZAP Express expression vector, U.S. Pat. No. 5.128.256). As an alternative, other vectors which are known to the person skilled in the art can be used, and particularly preferred are filamentous phage vectors, eukaryotic vectors, retroviral vectors, adenoviral vectors, or alpha virus vectors.

Screening and Identification of the Biologically Active Structure

The final step in the method according to the invention comprises screening the genomic expression bank and identifying the biologically active structure of the pathogen by the immunological response of infected hosts.

The term “biologically active structure” used here designates pathogen antigens, enzymatically active proteins, or pathogenity factors of the microbial pathogen.

According to a preferred embodiment of the present invention, screening represents an immuno-screening process for pathogen antigens, and the identification of pathogen antigens comprises the following steps: infection of bacteria with lambda phages, the cultivation of the infected bacteria with the formation of phage plaques, the transfer of the phage plaques onto a nitrocellulose membrane (or other solid phase suitable for the immobilisation of recombinants from the proteins derived from the pathogens), incubation of the membrane with serum or body fluids of the infected host containing antibodies, washing the membrane, incubation of the membrane with a secondary alkaline phosphatase-coupled anti-IgG antibody which is specific for immunoglobulins of the infected host, detection of the clone reactive with the host serum by colour reaction, and the isolation and sequencing of the reactive clones.

In principle, it is also possible, for the identification of the biologically active structure, to introduce pathogen nucleic acids into recombinant filamentous phage vectors (such as pJufo), which make possible an expression of antigens directly on the surfaces of the filamentous phages. In this case, the identification of pathogen antigens would encompass the following steps: generating recombinant filamentous phages by the introduction of filamentous phage vectors in bacteria, incubation of generated recombinant filamentous phages with serum of an infected host, selection of the filamentous phages to which the immunoglobulins of the host have bonded, by means of immobilized reagents which are specific to the immunoglobulins of the infected host, and the isolation and sequencing of the selected clones.

Proteins derived from the pathogen genome and expressed recombinantly can, for example, be bonded on the solid phase or screened within the framework of a panning/capture procedure with specific immunological response equivalents of the infected host. These are, on the one hand, antibodies from different immunoglobulin classes/sub-classes, primarily IgG. Host serum is used for this purpose. These, however, are also specific T-lymphocytes against epitopes of pathogen antigens, recognized as MHC-restringent, which must be tested in a eukaryotic system.

The conditions for establishing the genome bank are such that the inserted fragments occur according to the random generator principle due to the unique nature of the PCR primer. Accordingly, regions from known antigens are represented which are naturally also formed as proteins. Even fragments from intergenic regions which are normally not expressed can occur, which, depending on the length of open reading frames, can lead to the expression of short nonsense proteins or peptides. One important consideration is that, in the method according to the invention, even pathogen proteins which have not been identified hitherto can automatically be present.

Detailed Description of Preferred Embodiments of the Present Invention

The present invention combines the expression of the overall diversity of all conceivable recombinant proteins with the subsequent use of a highly stringent filter, namely the specific immunological response occurring in infected hosts within the framework of the natural course of the disease.

In a further embodiment of the invention, the pathogens are enriched prior to the amplification of the nucleic acids by precipitation with polyethylene glycol, ultra-centrifugation, gradient centrifugation, or by affinity chromatography. This step is not obligatory, however.

Precipitation with polyethylene glycol is efficient particularly with viral particles. As an alternative, with known pathogens, affinity chromatography making use of pathogen-specific antibodies against defined and stable surface structures is another option. A further alternative with unknown pathogens is the use of polyclonal patient serum itself, whereby the polyclonal patient serum is immobilized in the solid phase and used for affinity enrichment of pathogens as a specific capture reagent. The method described here can be used as a platform technology in order to identify highly efficient antigen-coding pathogen nucleic acids from very small amounts of material containing pathogens. As is shown in the examples below, 1 to 20 pg of genomic nucleic acid is sufficient to permit a comprehensive identification of the antigen repertoire of individual pathogens identified serologically by natural immunological responses. Thus, the technology described herein enabled, for example, the identification of antigens known to be immunodominant for vacciniavirus starting from 20 pg DNA.

The small quantity of nucleic acids required for this method makes it possible to apply the method for medically important questions which could only be dealt with in an unsatisfactory manner with the previously known methods, or not at all:

This includes, for example, the systematic direct identification of pathogen nucleic acids from infected cells, such as receptive in vitro cell lines, organs, inflammatory lesions such as pustules on the skin or mucous membranes, from infected internal lymphatic and non-lymphatic organs, or from fluids containing pathogens (such as saliva, sputum, blood, urine, pus, or other effiusions) obtained from infected organs. Taking as a basis a sensitivity of 1 pg, depending on the genome size of the pathogen (e.g. viruses 3,000-250,000 bp or bacteria 100,000-5,000,000 bp), 50 to 10⁵ pathogen particles are sufficient to identify pathogen nucleic acids coding for antigens. One may expect that in most cases the number of pathogen particles will be far above the sensitivity limit of the method according to the invention.

The high sensitivity of the method according to the invention allows, as already described above, an examination of the pathogens from primary isolates without the need for in vitro culturing. It is particularly important that very small quantities of source material, such as pinhead-sized biopsies or a few μL of infected sample fluids, are sufficient for the successful enrichment and identification of biologically active structures using the method according to the invention. Accordingly, the method according to the invention can be applied to any excess material from the field of medical-clinical diagnostics and to cryoarchived sample materials (as shown in Example 9).

The sequencing of the pathogen nucleic acids identified on the basis of the method according to the invention leads to the identification of the pathogen from which the nucleic acid originally came. Accordingly, prior knowledge of the pathogen is not required, and the method is suited for discovering previously unidentified infection pathogens or previously unknown antigens.

The method can therefore be applied to investigate a series of diseases in which the presence of an infection pathogen is etiologically suspected, but which could not yet be identified. This includes diseases which partially fulfil the Koch's postulate (such as communicability), but for which it has not yet been possible to identify the germs due to the lack of culturability/isolatability of the pathogens. Other examples are diseases such as sarcoidosis, pitrysiasis rosea, multiple sclerosis, diabetes mellitus, and Morbus Crohn.

Likewise, among a proportion of patients with etiologically unclear chronic hepatitis (chronic non-B, non-C hepatitis), a previously unknown viral disease is suspected. The germ counts in the serum of patients with known chronic viral hepatitis are high. For example, among patients with infectious chronic HBV-induced or HCV-induced hepatitis, in 1 mL of blood there are 10⁷-10⁸ hepatitis B or hepatitis C particles. On the assumption of an approximately comparable germ count, the method according to the invention is also suited for identifying putative non-B, non-C hepatitis pathogens using small volumes of blood/serum (1-10 mL) from infected patients.

The step of immunoscreening in the present invention, as a highly sensitive and highly specific high throughput detection method, makes it possible to identify an antigen-coding nucleic acid among 10⁶-10⁷ non-immunogenic clones.

For this purpose, a low degree of purity of the pathogen nucleic acids is sufficient for the identification of the pathogen. This low degree of purity can be attained with no difficulty by a variety of the methods known from the prior art, such as the precipitation of pathogen particles with polyethylene glycol (PEG) and/or affinity chromatography and/or degradation of contaminating host nucleic acids with nucleases.

An additional possibility for enriching pathogen particles is the use of the specific antibodies for capture processes formed in infected organisms against pathogen particles.

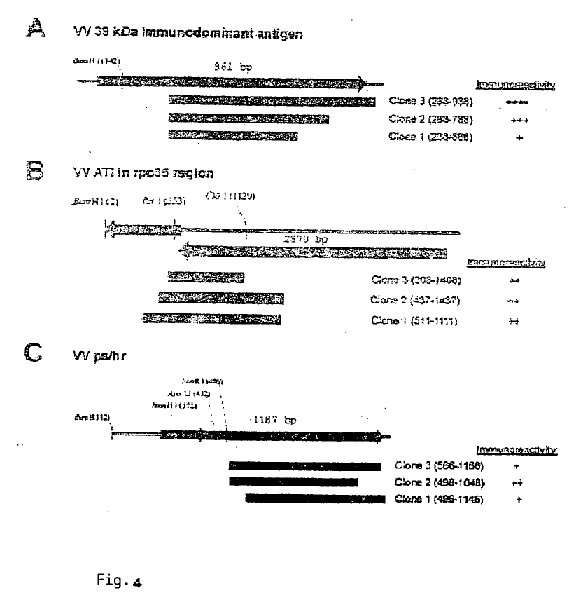

Immunoscreening as an integral part of the method allows the analysis of 10⁶-5×10⁶ clones within a short period of time (two months) by one single person. The combination of sequence-independent amplification and serological examination, with high throughput of all nucleic acid segments in all six reading frames, allows, even at moderate purity of the initial nucleic acids (pathogen nucleic acids >1% of the total nucleic acids), a comprehensive examination of all the regions potentially coding for polypeptides, regardless of the current expression status. By examining genomic nucleic acids, all gene regions will be covered, including genes which are only engaged at specific points in time (e.g. only in specific infection time phases). This not only makes it possible to make a statement on individual antigens, but also provides information about the whole of the nucleotide-coded immunogenic regions (immunome, see FIGS. 4A-C and 5). In addition, through the identification of multiple, partly overlapping fragments, it is possible to achieve a narrowing of the serologically recognized epitope within an identified antigen (see FIGS. 4A-C). The strength of the signal makes it possible to carry out further discrimination of dominant and non-dominant epitopes (see FIGS. 4A-C). The pathogen nucleic acid fragments identified are directly available for the development of immunodiagnostic agents and vaccines. The nucleic acid identified can be used as a matrix for the development of highly sensitive direct pathogen-detecting methods, for example by using nucleic acid-specific amplification by polymerase chain reaction (PCR). The fragments identified can also be used for the development of diagnostic tests based on the detection of the presence of antigen-specific T-lymphocyte reactions.

The way in which [the present method] differs from technologies such as “proteomics” has already been discussed in the preamble. The method according to the invention differs in technical terms from two other related methods used: serological investigation of genomic pathogen libraries and SEREX technology.

For the serological examination of genomic libraries, a number of groups (such as Luchini et al. (1983), Curr Genet 10:245-52, Bannantine et al. (1998), Molecular Microbiology 28: 1017-1026) have produced expression libraries from purified and mechanically chopped up or enzymatically digested pathogen DNA For the establishment of expression libraries with this method, in order to produce representative banks according to the size of the genome, between 0.5 and 5 μg of purified pathogen nucleic acids are required (factor 10⁵-10⁶ additional requirement for pathogen nucleic acids). Consequently, the method is not suitable for examining, pathogens from primary isolates in which far smaller quantities of pathogens and pathogen nucleic acids are present. It is essential in this situation that the pathogens be isolated, cultured in vitro, and then undergo a complex process of purification. Accordingly, as a basic prerequisite for using this method, the culturing and purification modalities must be known for each individual pathogen and established in advance. However, for many viruses and intracellular pathogens, this is a technically complex process requiring considerable expertise. This therefore also eliminates the possibility of identifying unknown pathogens, including those which are no longer live.

One must also distinguish this method from the method called SEREX (Sahin et al. (1995), Proc Nati Acad Sci USA 92: 11810-3; Sahin et al. (1997), Curr Opin Immunol 9, 709-716). For SEREX, mRNA is extracted from diseased tissue, cDNA expression libraries are established and screened for immunoreactive antigens with serums from the same individual from whom the tissue was taken.

A substantial difference between this and the method according to the invention lies in the fact that cDNA expression libraries from host cells of infected tissue are used for the SEREX method.

The differing quality and the differing origin of the nucleic acids lead to the following distinctions:

The use of total mRNA from host cells, using the SEREX method, increases the complexity of the library and reduces the probability of the identification of pathogen-derived transcripts. For animal host cells, one must assume the presence of 40,000 to 100,000 different host-specific transcripts. The number of transcripts for most pathogens is far lower (for viruses 3-200 transcripts, and for bacteria 500-4,000 potential gene products). In addition, in most cases only a small proportion of the host cells are infected with pathogens, with the result that the portion of pathogen-derived nucleic acids in the total mRNA population is further diluted. Because a large number of host-specific transcripts also code for natural or disease-associated autoantigens (Sahin et al., 2000, Scanlan et al. (1998) Int J Cancer 76, 652-658), the identification of pathogen-derived antigens by the preferential detection of host tissue autoantigens is made very difficult. This is responsible for the fact that hitherto no pathogen antigens have been identified using the SEREX technology when examining a number of infected tissue samples, such as HBV-Ag+ liver cell carcinomas (Scanlan et al. (1998), Int J Cancer 76, 652-658; Stenner et al. (2000), Cancer Epidemiol Biomarkers Prev 9, 285-90).

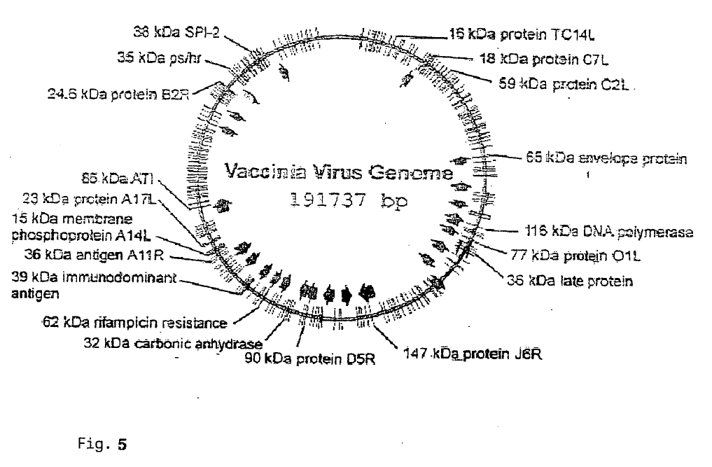

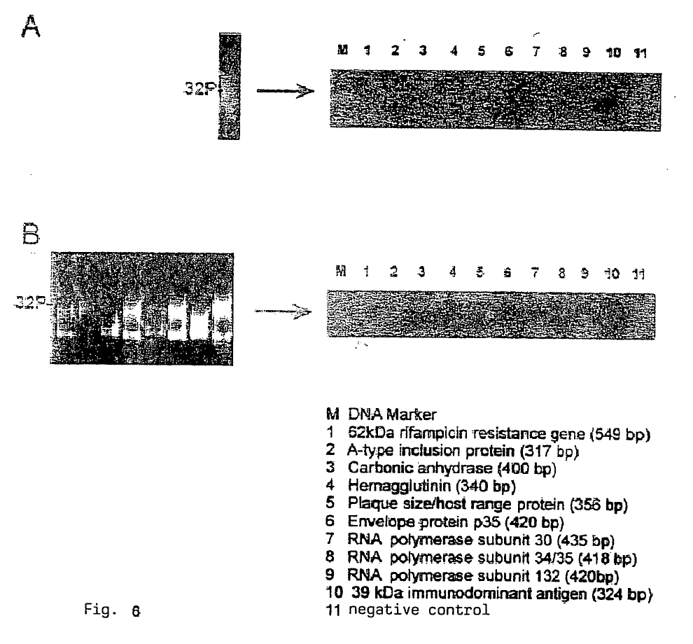

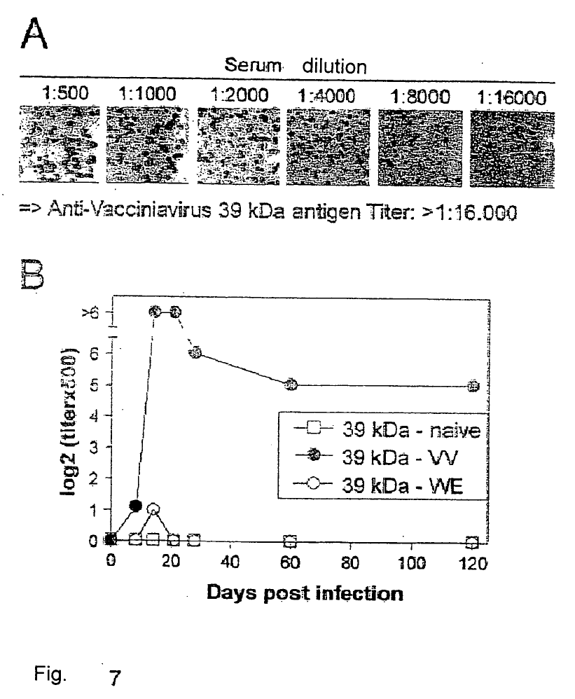

From the following illustrations and examples it can be seen that with the method according to the present invention it is possible to identify and characterize immunologically relevant viral and bacterial antigens from extremely small quantities of pathogen nucleic acids. The viral antigens identified in the following examples were in this case distributed over the entire genome of the vaccinia virus, which allows us to conclude that there is a satisfactory representation of the different genes in the DNA amplified with the aid of the method according to the invention (see FIG. 5). One of the antigens identified in the examples even evokes neutralizing antibodies and is therefore of great significance in the therapeutic context. Accordingly, the method is also suited for detecting antigens important for therapy.

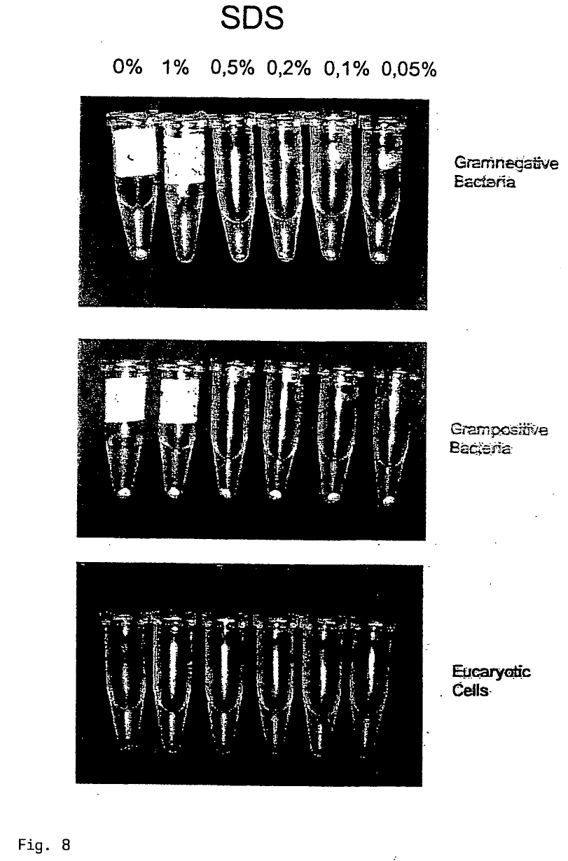

The method according to the invention was used in the present examples for the identification of viral and bacterial antigens. According to the invention, the following SDS concentration is preferably used for bacterial pathogens for the enrichment of gram-negative and gram-positive pathogens respectively (see FIG. 8): >0.05% to 1% for gram-positive bacterial and 0.05% to 0.1% of SDS for gram-negative bacteria.

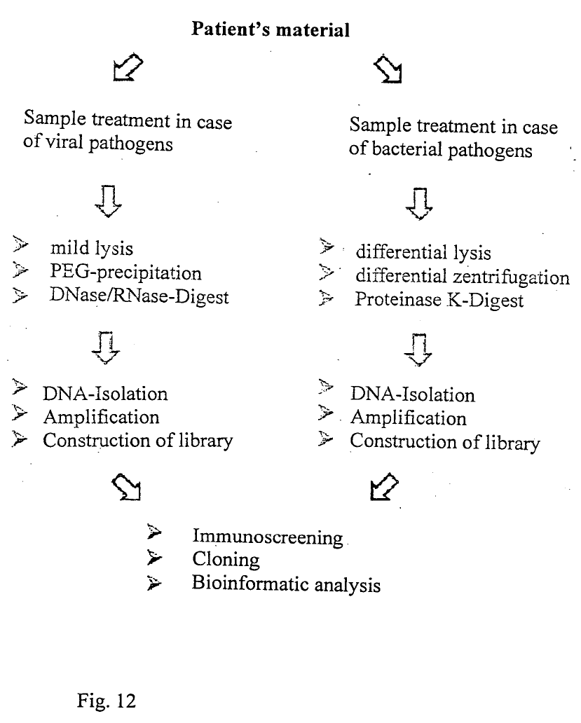

If there is no indication of whether a pathogen is a virus or a bacterium, the initial sample, because of the very small material quantity required (e.g. into two sample vessels), can be divided up and processed using different methods on the assumption of a causative viral or bacterial pathogen (FIG. 12).

Another feature of the present invention concerns new vaccinia virus antigens, characterized in that the antigen is coded by a nucleic acid which exhibits 80% homology, in particular 90% homology, and preferably 95% homology in one of the sequences SEQ ID NOS: 4,5,6,7,8,9,10,11,12,13,14,15,16,17,18,19,20,21 or 22.

Particularly preferred are vaccinia virus antigens which are characterized in that the antioen is coded by one of the nucleic acids SEQ ID NOS: 4,5,6,7,8,9,10,11,12,13,14,15,16,17,18,19,20,21 or 22.

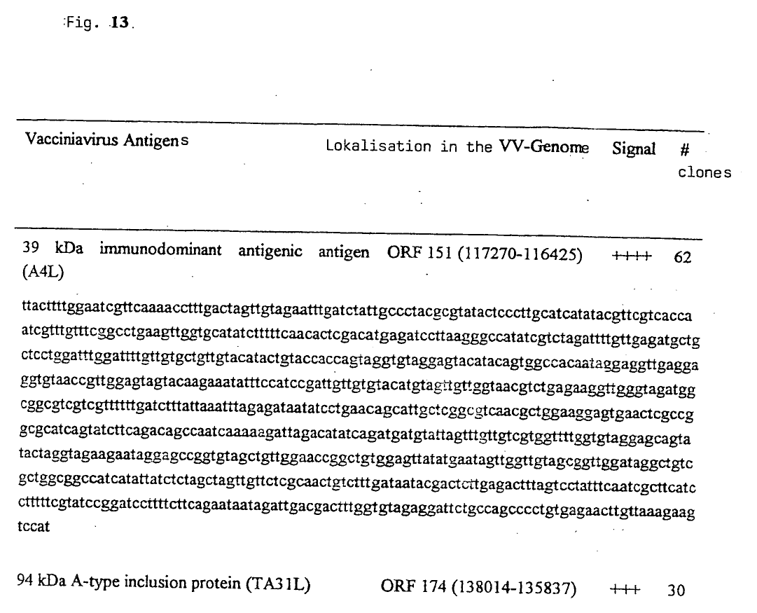

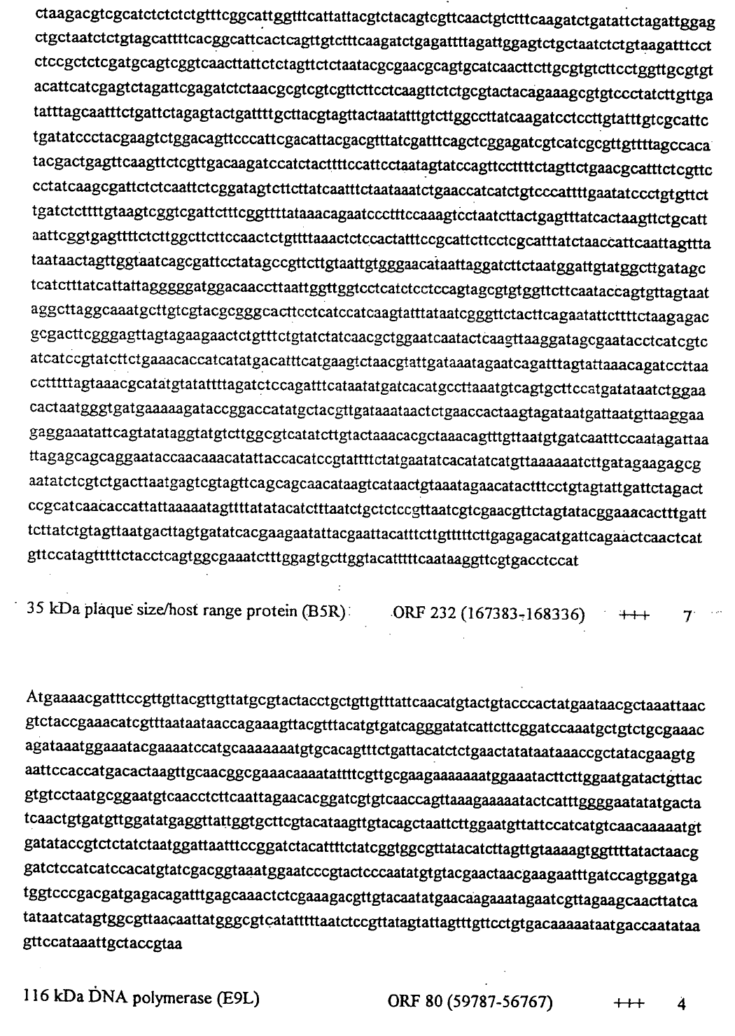

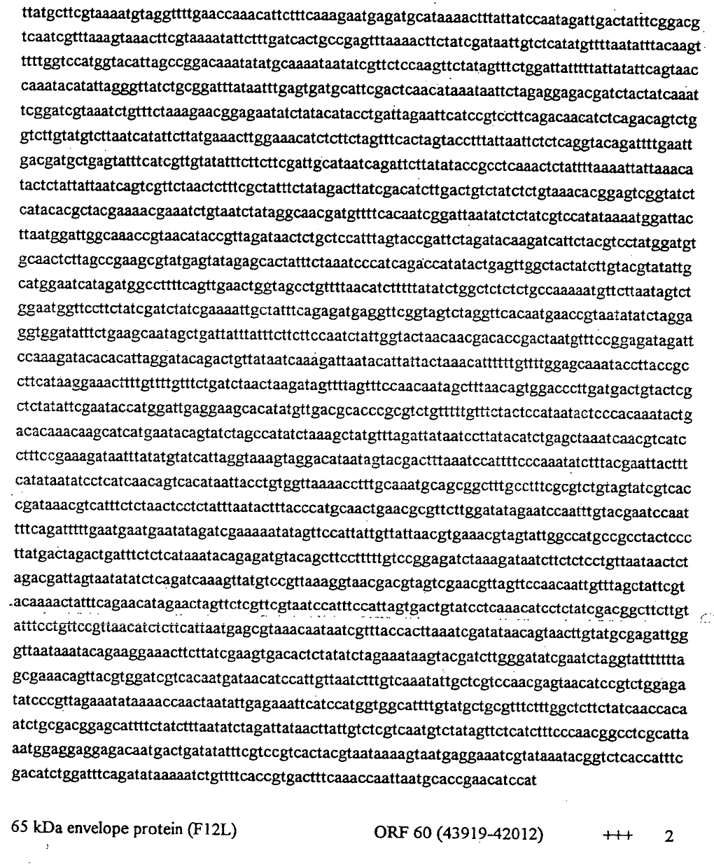

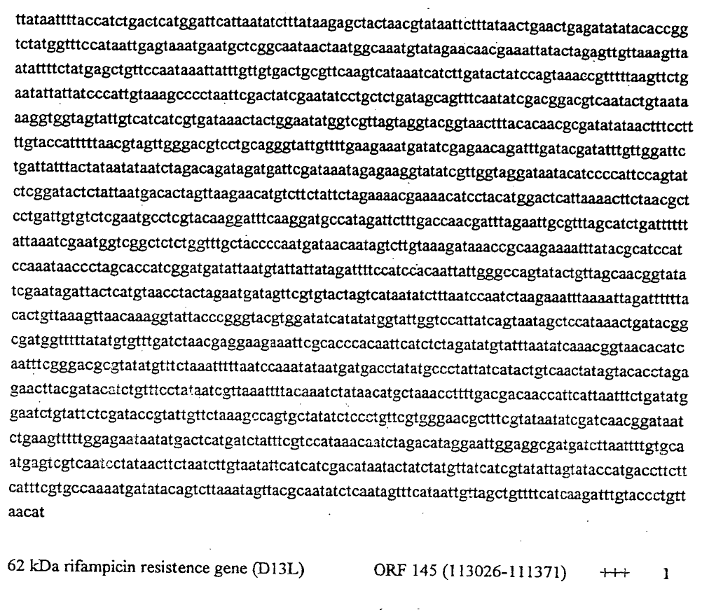

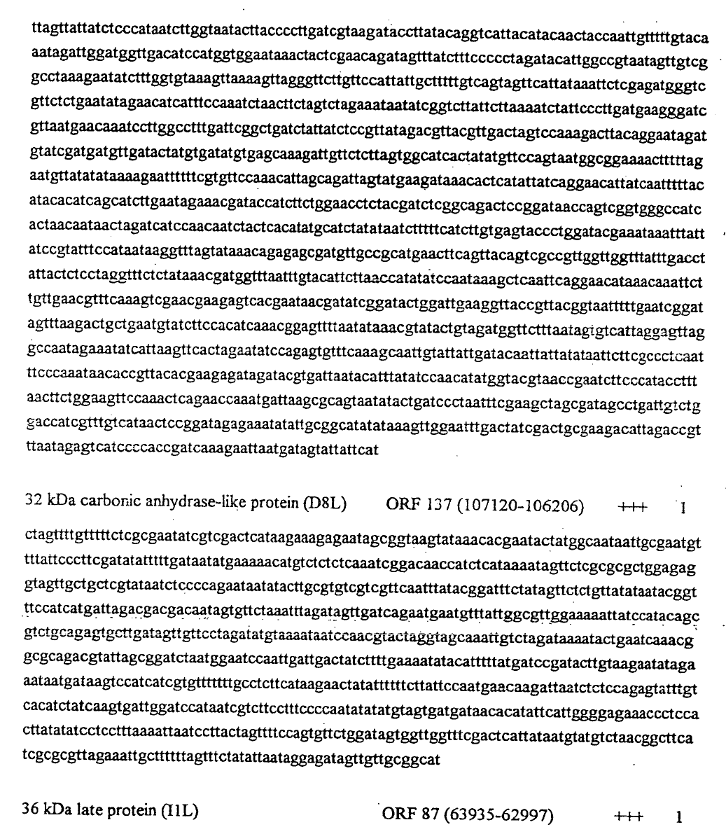

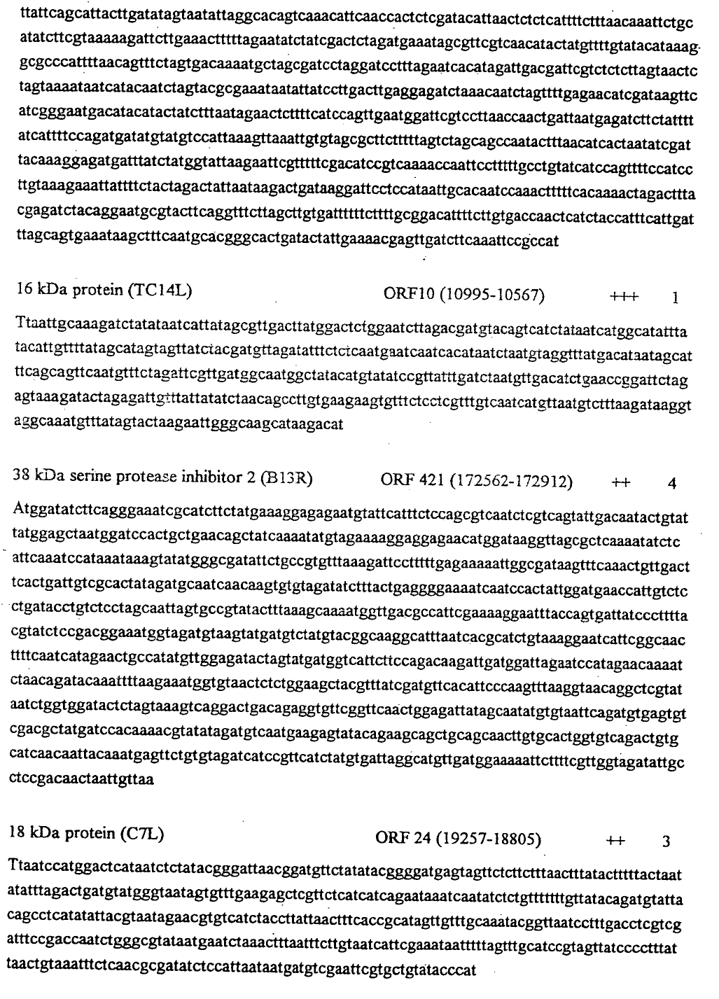

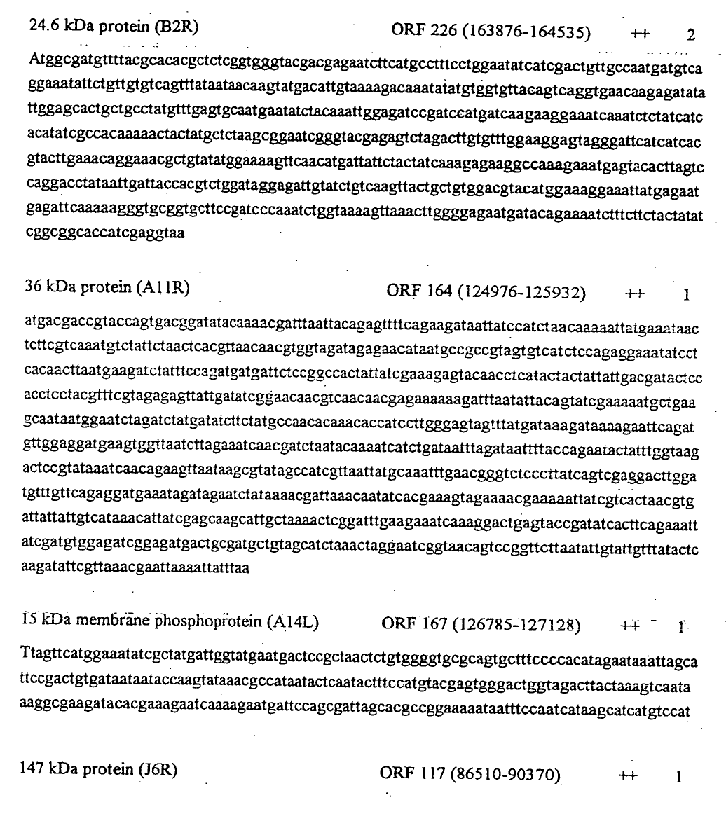

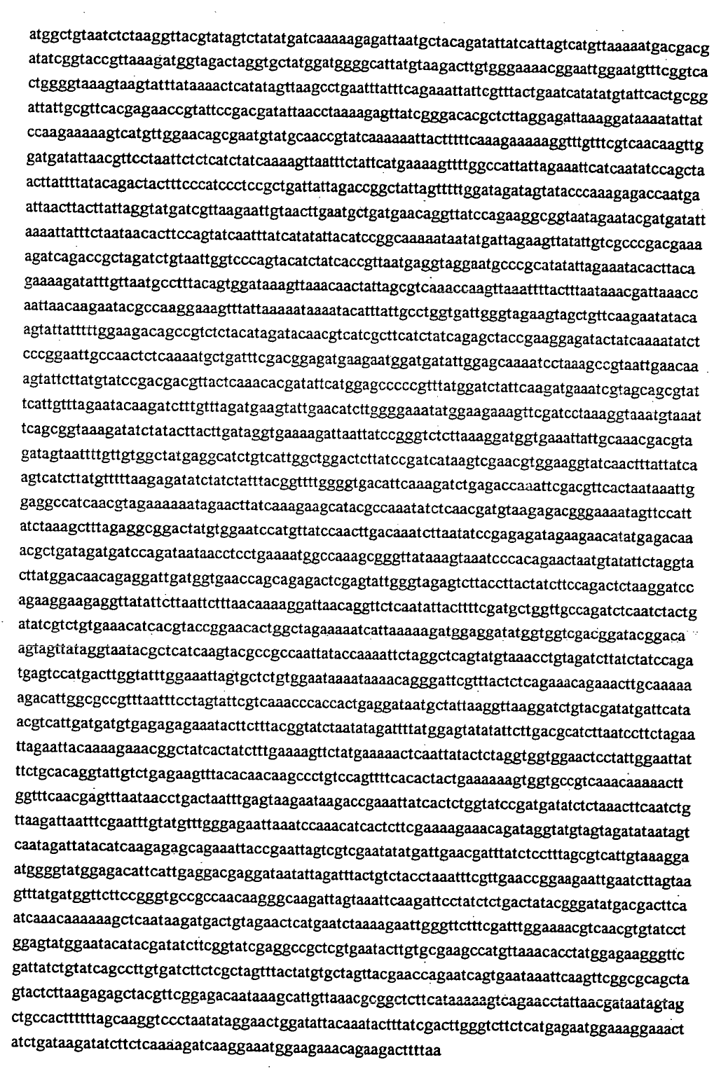

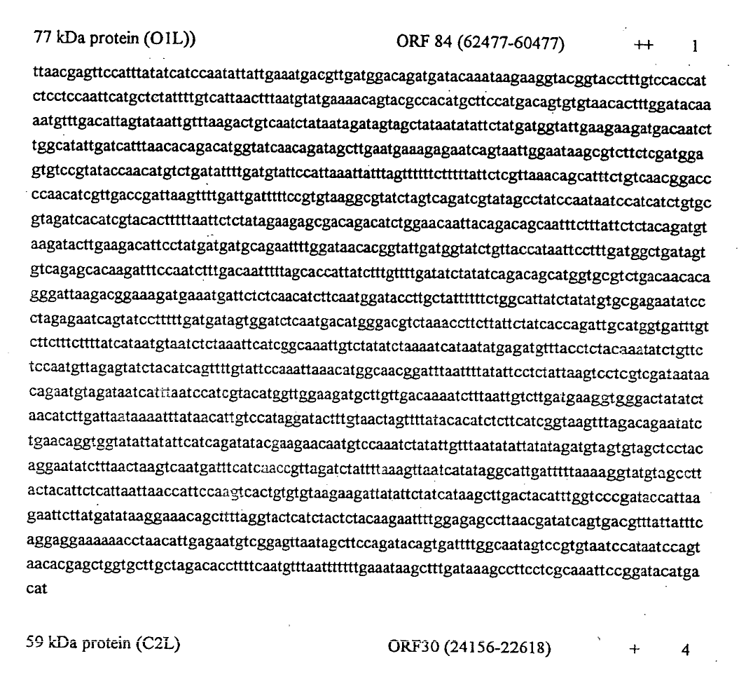

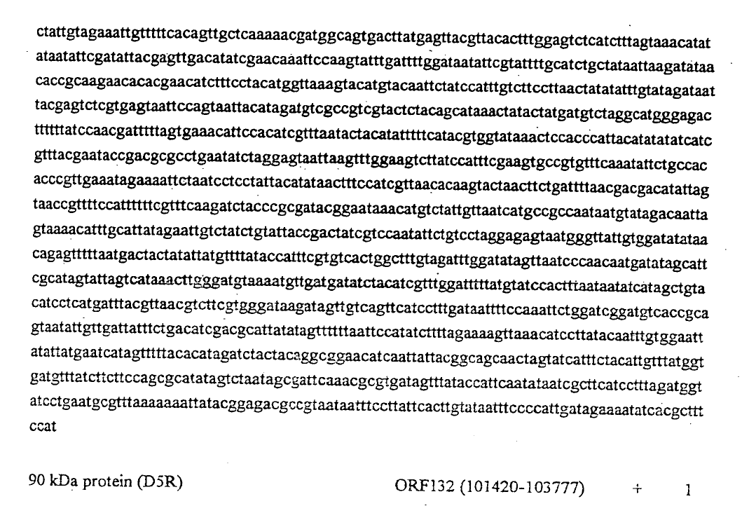

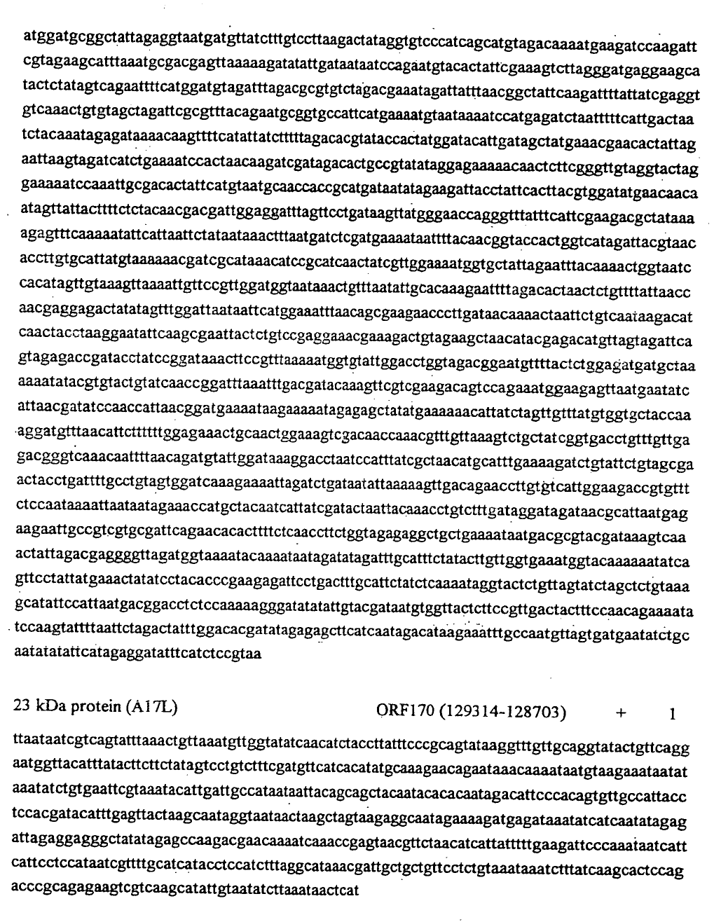

The vaccinia virus antigens according to the invention, which are identified by the method according to the invention, are described in greater detail in Example 5 and Table 3. Preferred nucleic acid sequences which code the vaccinia virus antigens are represented in the sequence protocol as SEQ ID NOS: 4,5,6,7,8,9,10,11,12,13,14,15,16,17,18,19,20,21 or 22. The preferred nucleic acid sequences are also represented in FIG. 13.

Another feature, therefore, also concerns the use of the nucleic acids SEQ ID NOS: 4-22 and of the nucleic acids which exhibit 80%, 90%, or 95% homology with the former nucleic acids, in the methods for detecting the vaccinia virus. Such detection methods are known to the person skilled in the art.

Further uses for the vaccinia virus antigens according to the invention are:

Possible serological detection of Variola major (smallpox pathogen) by means of conserved epitopes in vaccinia virus antigens. Variola major is not available for immunological analysis, since it is secured in military laboratories.

Vaccination against variola major with sub-unit vaccines. Vaccination with the vaccinia virus provides good protection against the smallpox pathogen. Individual vaccinia virus antigens are therefore potential candidates for the induction of immunological protection against the smallpox pathogen.

The following figures serve to explain the invention:

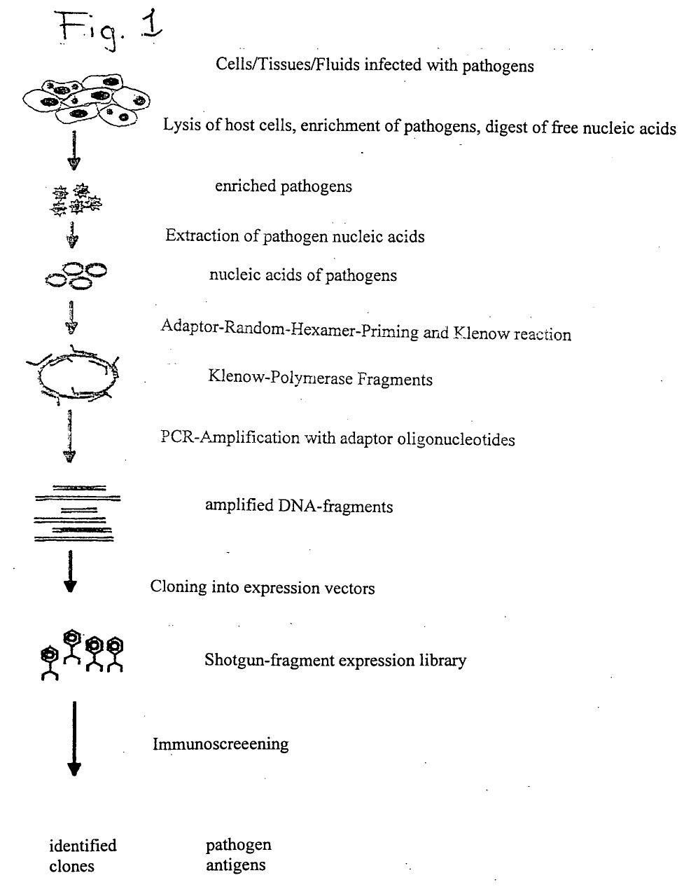

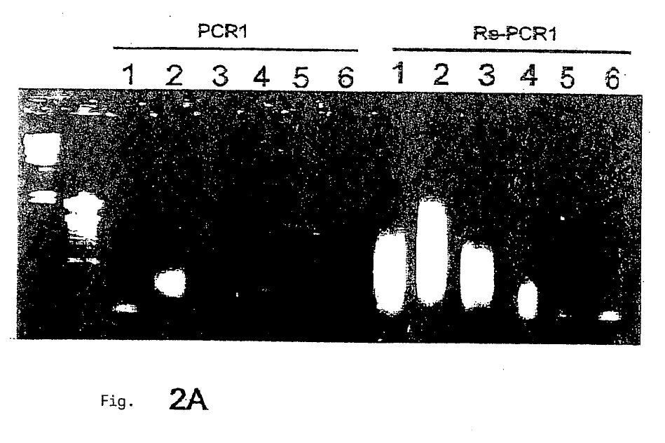

FIG. 1. Schematic representation of the sequential analytical steps of an embodiment of the method according to the invention.

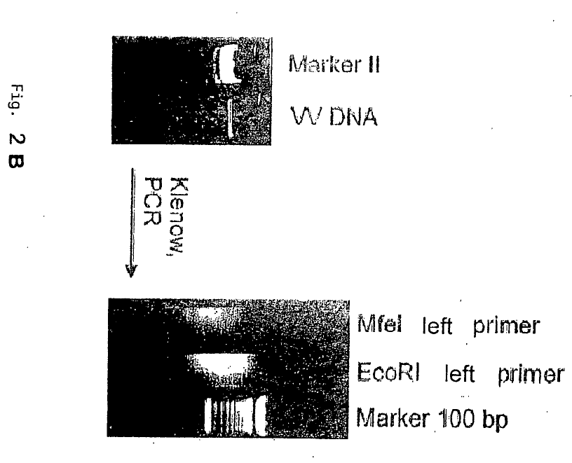

FIG. 2A. Amplification of different source quantities of pathogen nucleic acids.

Klenow-tagged vaccinia virus DNA, as explained in Example 3A, was amplified once for 35 cycles (PCR1) and 1 μL was re-amplified for a further 35 cycles (Re-PCR1). 1 ng (Lane 1), 40 pg (Lane 2), 8 pg (Lane 3), 0.8 pg (Lane 4), and 0.08 pg (Lane 5) respectively of Klenow enzyme-tagged vaccinia virus DNA were used as source quantities for the armplification. Lane 6 represents the negative control without the addition of vaccinia virus DNA.

FIG. 2B. Amplification of vaccinia virus DNA; the amplification of vaccinia virus DNA was induced by a Klenow enzyme reaction (left), for which adaptor oligonucleotides with a degenerated 3′ end were used for sequence-independent priming. This was then followed by the actual PCR amplification with oligonucleotides corresponding to the adaptor sequence. The PCR conditions described produced fragments of different lengths (200-2500 bp) (right).

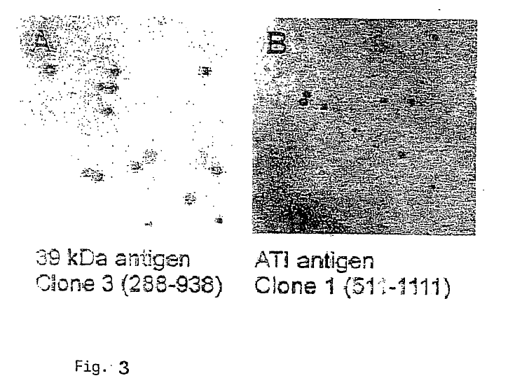

FIG. 3. FIG. 3 shows the immunoscreening and identification of the 39kDa antigen clone 3 (288-939) and the ATI antigen clone 1 (511-111). Clones initially identified in the screening were then isolated oligoclonally by including adjacent non-reactive phage plaques and were rendered monoclonal after confirmation.

FIG. 4A. FIG. 4A shows clone 1 (288-688), clone 2 (288-788), and clone 3 (288-938), which code for overlapping regions from the 39kDa protein of the vaccinia virus. The clones are differently immunoreactive.

FIG. 4B. FIG. 4B shows three clones which code for overlapping ranges of the A-type inclusion protein (ATI) of the vaccinia virus and exhibit the same immune reactivity.

FIG. 4C. FIG. 4C shows three clones which code for the overlapping ranges of the plaque size/host range protein (ps/hr) of the vaccinia virus and are differently immunoreactive.

FIG. 5. FIG. 5 shows the distribution of the clones in the vaccinia virus genome identified according to the invention. The identified antigens are distributed over the entire vaccinia virus genome, which allows the assumption that there is satisfactory representation of the vaccinia virus gene in the library established by means of the method according to the invention.

FIG. 6. FIG. 6 shows the molecular analysis of the representation of ten arbitrarily selected vaccinia virus genes in the vaccinia virus DNA amplified by the method according to the invention. Ten gene segments from the genome of the vaccinia virus were synthesized by PCR. Amounts of 10 ng each of the gene segments, 317 to 549 bp long, were separated in agarose gels by means of gel electrophoresis, and transferred onto a nylon membrane using the Southern blot method. For producing the ³²P-marked probe, 10-20 pg of vaccinia virus DNA were used. FIG. 6A shows the hybridization with PCR fragments from a single Re-PCR. FIG. 6B shows the improvement of the representation due to the fact that, for the hybridization, pooled fragments from several Re-PCR's varied as described in Example 2 were used. The hybridization of the pooled amplified DNA with the ten blotted and randomly selected segments (visible as weak to clear blackening) of the vaccinia virus genome shows that all the segments are contained in the amplified DNA. It must be emphasized that even the gene with the weakest hybridization signal (Lane 2, 94 kDA A-type inclusion protein, ATI) was identified 30 times as an antigen in the immunoscreening of the library (see Table 3).

FIG. 7A. FIG. 7A shows the determination of the serum titer against the immunodominant 39 kDa antigen of the vaccinia virus cloned by the method according to the invention. Serum from C57/BL6 mice was drawn on day 21 after infection with the vaccinia virus and diluted as indicated. For the production of the antigen, E. coli bacteria were infected with a lambda phage coding the 39 kDa antigen. The reactivity of the serum dilutions against the 39 kDa antigen recombinantly expressed in this manner was tested on nitrocellulose membranes. For the serum used here, the antibody titer is >1:16000.

FIG. 7B. FIG. 7B shows the curve of the antibody titer against the 39 kDa antigen after infection with 2×10⁶ pfu of vaccinia virus or with 2×10⁵ pfu of the lymphocytary choriomeningitis virus (WE strain). Non-infected titers (naive) show no reactivity against the 39 kDa antigen. The high specificity of the reaction is also shown by the only minimal cross reactivity with the serum from the mice infected with the lymphocytary choriomeningitis virus (day 14).

FIG. 8. FIG. 8 shows the different sensitivity of eukaryotic cells and gram-negative and gram-positive bacteria respectively. For the experiment, comparable cell volumes of gram-negative bacteria (top), gram-positive bacteria (middle), and eukaryotic cells respectively were incubated with the concentrations of SDS indicated. Non-lysed corpuscular structures were pelleted by centrifugation. While eukaryotic cells are already fully lysed by SDS at minimal concentrations (no visible cell pellet and no microscopically visible cells), bacteria are more resistant and, because their corpuscular integrity is maintained, can be enriched by centrifugation.

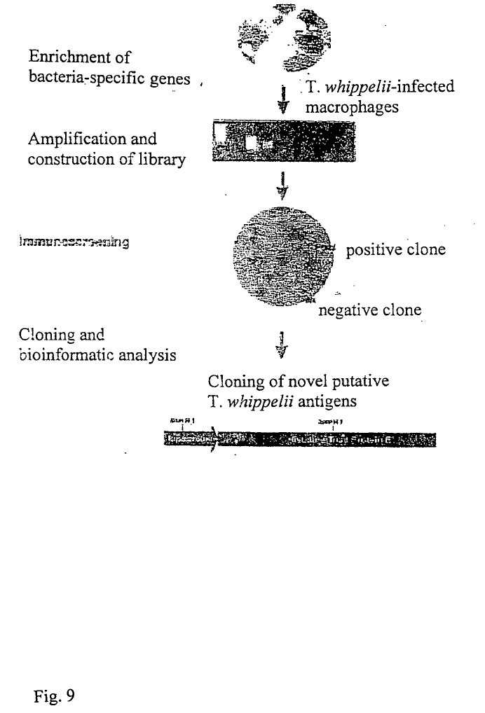

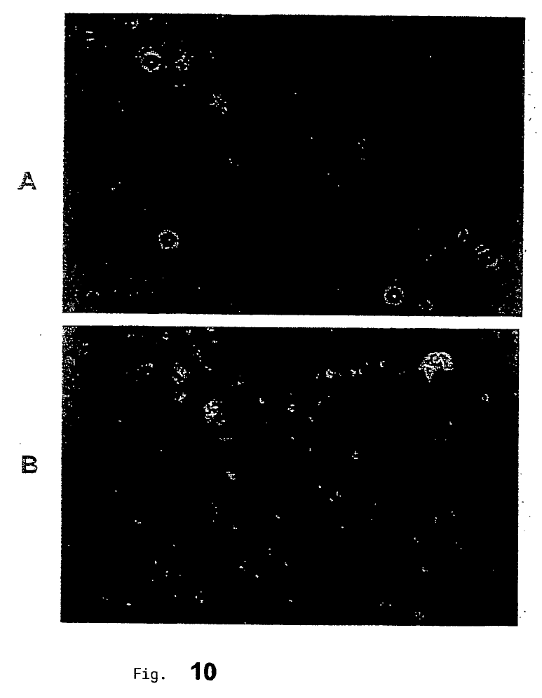

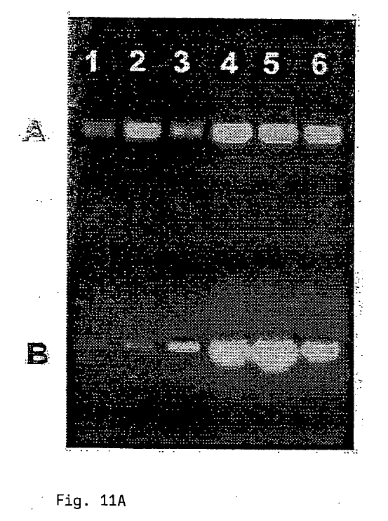

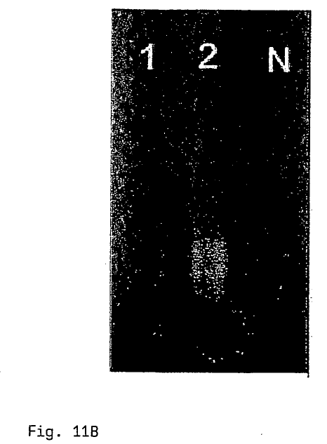

FIG. 9. FIG. 9 shows the identification and molecular characterization of putative antigens of the human pathogenic bacterium Tropheryma whippelii. Interleukin-10 and Interieukin-4 deactivated human macrophages were incubated with brain material containing T. whippeli. bacteria taken from a patient who had died of Whipple's disease. Bacteria-specific genes were isolated by differential lysis and subsequent DNA processing, and libraries were established using the method according to the invention. The immunoscreening was carried out with sera from patients infected with T. whippelli. The bioinformatic analysis, i.e. the comparison with publicly accessible sequence databases, shows that hitherto unknown antigens were identified by the method according to the invention.

FIG. 10: Isolation of bacteria directly from the spleen tissue of a patient with Whipple's disease. Bacteria, as shown in Example 9, were isolated from a cryopreserved spleen sample from a patient with Whipple's disease and analyzed by fluorescence microscopy. The image shows the superposition of the exposure in phase contrast (proof of corpuscular particles, upper part) and following superposition with a blue fluorescence signal (proof of DNA).

FIG. 11A: Enrichment of pathogen nucleic acids coming directly from a patient sample as described in Example 9.

FIG. 11B: Amplification of pathogen nucleic acids isolated directly from patient samples. The bacterial DNA enriched from the spleen sample was amplified as described in Example 10 (Lane 2). Lane 1 shows the positive control with another DNA sample. The negative control without additional DNA is applied to Track 3.

FIG. 12: Diagram of a possible procedure for identifying pathogen antigens when it is not known whether the pathogen is a virus or a bacterium. The initial sample is separated and processed by means of different methods allowing the identification of bacterial and/or viral pathogens.

FIG. 13: Nucleic acid sequences of the identified vaccinia virus antigens listed in Table 3. The nucleic acid sequences correspond to the sequences SEQ ID NO: 4 to SEQ ID NO: 22 in the sequencing protocol.

The present invention is also illustrated, in a non-limitative manner, by the following examples.

### EXAMPLES

### Example 1

Isolation of Pathogen Nucleic Acids from Virus-infected Cells:

BSC40 cells were infected with 2×10⁶ pfu of vaccinia viruses. The infected cells were incubated for 24 hours at 37° C. in a CO₂ incubator and then harvested. The harvested cells were then homogenized and absorbed in a buffered medium. To separate virus particles from host cell fragments, the cell lysate absorbed by the medium was then treated with ultrasound. Coarse particulate structures were pelleted by centrifugation for 15 min at 3000 rpm. Following centrifugation, the supernatant was removed and the pellet discarded. In order to precipitate corpuscular particles in the supernatant, 2 mL of cooled supernatant were precipitated with 6% PEG6000/0,6M NaCl (1 h incubation on ice); the precipitate was then pelleted for 10 min by centrifugation at 10000×g. As the preceding steps should have yielded both a separation and a lysis of contaminating host cells, the precipitate was then absorbed into 300 μL of DNAse/RNAse buffer after discarding the supernatant, and digested with RNAse und DNAse for 30 min at 37° C. This brought about the elimination of the now extracellular nucleic acids of the previously disintegrated host cells. The virus nucleic acids are protected from the nucleases by the intact virus capsid and not degraded. Virus particles were broken down by vortexing with 1 volume of GITC buffer, which also inactivates the added nucleases. Released pathogen DNA was extracted with phenol/chloroform and precipitated with 1 iso-volume of isopropanol. The precipitated nucleic acids were washed with 80% ethanol and absorbed into 20 μL of distilled H₂O.

### Example 2

Infection with Vaccinia Virus and Obtaining Serum

Recombinant vaccinia virus with the glycoprotein of the vesicular stomatitis virus (VaccG) (Mackett et al. (1985), Science 227, 433-435.) was cultured on BSC40 cells and the virus concentration determined in a plaque assay. C57BL/6 mice (Institute of Laboratory Animal Science, University of Zurich) were infected intravenously with 2×10⁶ pfu of VaccG. Blood samples of 200-300 μl were taken from the mice on days 8, 16 and 30 following infection, and serum was obtained through centrifugation and stored at −20° C.

After the mice had been immunized, the successful induction of anti-vaccinia antibodies was tested against VSV in a neutralization assay (Ludewig et al. 2000. Eur J Immunol. 30:185-196) to determine the best time to extract serum for the planned analyses. As shown in Table 1, the increase of both the total immune globulin and the IgG class reached its maximum as of day 16, so that this serum extracted on days 16 and 30 could be used.

TABLE 1
Increase and decrease of titer of antibodies
following immunization with vaccinia virus

| | Total immune globulin | IgG |
|---|---|---|
| Day after inoculation | (in 40 × log2) | (in 40 × log2) |
| 8 | 9 | 6 |
| 16 | 12 | 11 |
| 30 | 12 | 12 |

### Example 3A

Global Amplification of Minimai Amounts of Pathogen Nucleic Acids

The global amplification of genomic nucleic acids of the pathogen is an essential step in the procedure according to the invention. The main challenge in this is to amplify in a comprehensive manner (i.e. including all the segments of the genome, if possible) the very small amount of genomic germinal nucleic acids which are isolated (without pre-culturing) from infected tissue. The amplified DNA must also be expressable and clonable for subsequent screening. While PCR-amplified cDNA-expression libraries are often described, produced and used (e.g. Edwards et al., 1991) and are sometimes commercialized as kits (e.g. SMART-cDNA library construction kit, Clontech), the establishment of comprehensive genomic libraries based on small amounts of pathogen nucleic acids (<10 ng) has hitherto not been described. It was therefore necessary to develop a DNA amplification module for the method according to the invention that would allow the production of comprehensive genomic expression banks from sub-nanogram material. The method was established for the vaccinia virus genome and can be transferred without modification to all DNA-coded pathogens and, with minor modification, to RNA-coded pathogens. The amplification of the vaccinia virus DNA was initiated by a Klenow enzyme reaction for which adaptor oligonucleotides with degenerated 3′ end were used for sequence-independent priming according to the random principle. This is followed sequentially by two PCR amplifications with oligonucleotides corresponding to the adaptor sequence. The conditions for an especially efficient amplification have been elaborated in a series of independently performed experiments. Following the optimization of the method, to determine the sensitivity of the method, different amounts of vaccinia virus DNA (e.g. 25 ng, 1 ng, 200 pg, 20 pg, 2 pg) were mixed with 2 pMol of Adaptor-N(6) (GATGTAATACGAA[P2] [P3]TTGGACTCATATANNNNNN), denatured for 5 min at 95° C. and then cooled on ice. N, in this context, is the degenerated primer portion. Following preparation of the primer reaction starter using Klenow's enzyme (2 U), DNA polymerase-1 buffer (10 mM of Tris-HCl pH 7.5, 5 mM of MgCl₂, 7.5 mM of dithiothreitol, 1 nMol of dNTPs), we carried out a primer extension for 2 h at 37° C. The fragments elongated through Klenow's polymerase were then purified of the free adaptor oligonucleotides using standard techniques. One 25th (i.e. 1 ng, 40 pg, 8 pg, 0,8 pg, 0,08 pg) each of the DNA tagged with Klenow's polymerase were used for a first amplification step. PCR amplification with adaptor oligonucleotides was performed with two different oligonucleotides (EcoR1 adaptor oligonucleotide GATGTAATACGAATTCGACTCATAT and/or Mfe1 adaptor oligonucleotide GATGTAATACAATTGGACTCATAT) (annealing at 60° C. for 1 min; extension at 72° C. for 2.5 min; denaturation at 94° C. for 1 min; 35 cycles). Single amplification of the nucleic acids for nucleic acids of less than 40 pg turned out not to be sufficient to produce an amplification smear which is optically detectable in the ethidium bromide/agarose gel. 1 μL each of the amplificate were therefore transferred as templates to a second amplification under identical conditions for 30-35 cycles. The amplified products were analyzed by gel electrophoresis. The described PCR conditions caused an amplification with fragments of different lengths (150-2000 bp) in all assay conditions down to a minimum of 0.8 pg of template DNA (FIG. 2A). In this process, shorter fragments on the average were amplified when initial amounts of DNA were lower. The conditions for Re-PCR were varied in different experiments. It turned out that varying the buffer conditions (e.g. Mg concentration) and the enzymes used, e.g. the Stoffel fragment of the Taq polymerase, produced different amplification patterns (see FIG. 6). Only 1/50 of the initial PCR was used for reamplification. Reamplification can therefore be performed under 50 different conditions. It was shown in a representative analysis in reverse Southern blot (see FIG. 6) that varying the amplification conditions allows a particularly satisfactory comprehensive global amplification. In order to verify the identity of amplified fragments, the latter were ligated through standard procedures in the blue-script cloning vector (Stratagene) and 20 clones were sequenced. 20 out of 20 sequences were identical with vaccinia virus sequences, so that an amplification of artifact sequences (e.g. polymerized primer sequences) was ruled out.

### Example 3B

Establishment of a Genomic Library

A vaccinia library was established by arnplifying 20 pg of vaccinia virus DNA tagged with Klenow's polymerase in analogy to the conditions in Example 3A. 1/50 each of the purified fragments (annealing at 60° C. for 1 min; extension at 72° C. for 2.5 min; denaturation at 94° C. for 1 min; 35-40 cycles) were used in separate solutions with two different oligonucleotides (EcoR1 adaptor oligonucleotides GATGTAATACGAATTCGACTCATAT and/or Mfe1 adaptor oligonucleotides GATGTAATACAATTGGACTCATAT) for PCR amplification with adaptor oligonucleotides. 1 μL each of the amplificate were subsequently transferred as template to a second amplification for 30 cycles under identical conditions. The amplified products were analyzed by gel electrophoresis. The described PCR conditions caused an amplification with fragments of different lengths (200-2500 bp) (FIG. 2B). All control reactions in which no DNA template was used remained negative. The amplified products were then purified, digested with EcoR1 and/or Mfe1 restriction enzymes and ligated in a lambda ZAP Express vector (EcoR1 fragment, Stratagene). Combining two independent restriction enzymes increases diversification and the probability that immunodominant regions are not destroyed by internal restriction enzyme interfaces and thereby remain undetected. Following the ligation of the nucleic acid fragments into the vectors, the latter were packaged in lambda phages using standard procedures. This was done with commercially available packaging extracts according to the manufacturers' instructions (e.g. Gigapack Gold III, Stratagene). The lambda phage libraries established in this fashion (SE with EcoRI adaptors, SM for MfeI adaptors) were analyzed without firther amplification by immunoscreening.

### Example 4

Immunoscreenine and Identification of Antigens

Immunoscreening was performed as described by Sahin et al. (1995), Proc Natl Acad Sci USA 92: 11810-3; and Tüeci et al. (1997), Mol Med Today 3, 342-349.

Bacteria from the E.coli K12-derived XL1 MRF strain were harvested in the exponential growth phase, set to OD₆₀₀=0.5 and infected with lambda phages from the described expression bank. The number of plaque-forming units, pfu, was set in such a way as to obtain a subconfluence of the plaques (e.g. ˜5000 pfu/145 mm Petri dish). By adding TOP agar and IPTG, the infection batch was plated on agar plates with tetracycline. In the overnight culture at 37° C., phage plaques formed on the bacterial lawn. Each individual plaque represents a lambda phage clone with the nucleic acid inserted into this clone and also containing the protein coded by the nucleic acid and expressed recombinantly.

Nitrocellulose membranes (Schleicher & Schuüll) were applied to produce replica preparations of the recombinant protein (plaque lift). Following wash steps in TBS/Tween and blocking of unspecific binding sites in TBS+10% milk powder, incubation occurred overnight in the serum of the infected host. Pooled serum from infection days 16 and 30 was used and diluted at 1:100-1:1,000 for this purpose. Following additional wash steps, the nitrocellulose membranes were incubated with a secondary AP-conjugated antibody directed against mouse IgG. In this manner, it was possible, by means of colour reaction, to make binding events of serum antibodies to proteins recombinantly expressed in phage plaques visible. It was thus possible to trace back clones identified as reactive with host serums to the culturing plate and, from there, to isolate the corresponding phage construct monoclonally. Such positive clones were confirmed following another plating. The lambda phage clone was recircularized to a phagemid by in vivo excision (Sahin et al. (1995), Proc Natl Acad Sci USA 92: 11810-3).

A total of 150,000 clones were screened in the way described above in the two banks (SE and SM). For this purpose, the pooled serum from the infected animals from day 16 and 30 following infection was used in a 1:500 dilution. Primarily identified clones were first isolated oligoclonally by including neighbouring non-reactive phage plaques and, after confirmation, monoclonalized (FIG. 3). 26 (SE bank) and/or 41 (SM bank) clones that were reactive with the serum from the immunized animals were isolated.

In addition, all identified clones were tested with pre-immune serums of the same strain from the mice; they were not reactive.

TABLE 2
Number of reactive clones after screening of the SE und SM bank

| Bank | Screened clones | Reactive clones |
|---|---|---|
| SE bank | 150,000 | 26 |
| SM bank | 150,000 | 41 |

Sequencing and comparing data bases uncovered, among other things, the three following differently immunogenic vaccinia virus proteins among the clones:

39 kDa Immunodominant Antigen Protein

Three clones code for segments from the 39-kDa protein of the vaccinia virus (FIG. 4A). The clones represent fragments of this protein. All of them start at nucleotide position 288, but extend at different distances to the 3′ end of this gene product, i.e. until nucleotide position 688, 788 or 938.

The gene coding for the 39 kDa protein is ORF A4L in the Western Reserve (WR) strain (Maa and Esteban (1987), J. Virol. 61, 3910-3919). The 39 kDa protein having a length of 281 amino acids is strongly immunogenic both in humans and animals (Demkovic et al. (1992), J. Virol. 66, 386-398).

It has already been described that immunizing with 39 kDa protein can induce protective immunity in mice (Demkovic et al. (1992), J. Virol. 66, 386-398).

The strongest antigenic domain seems to be within the last 103 amino acids located C-terminally (Demkovic et al. (1992), J. Virol. 66, 386-398). The position of the fragments found here is also to be considered as indicative of sero-epitopes. Interestingly, the two strongly immunoreactive clones 2 and 3 cover the region of these 103 amino acids, which are described as strongly antigenic.

This example also highlights the multidimensionality of the statements made on the basis of the method according to the invention. In addition to the identification of the antigen, which, at the same time, also provides immune protection, the number of overlapping clones is an indication of the abundance of the antibodies. The position of the clone allows the narrowing of the sero-epitopes, and the strength of the reactivity indicates the avidity of the antibodies. This also applies to the antigens described below.

A-type Inclusion Protein (ATI)

Some of the clones found here represent the A-type inclusion protein (ATI) (FIG. 4B), an approx. 160 kDa protein in various orthopox viruses (Patel et al. (1986), Virology 149, 174-189), which accounts for a large portion of the protein of the characteristic inclusion bodies. In the case of the vaccinia virus, this protein is truncated, its size being only 94 kDa (Amegadzie (1992), Virology 186, 777-782). ATI associates specifically with infectious intracellular mature vaccinia particles and cannot be found in enveloped extracellular vaccinia viruses (Uleato et al. (1996), J. Virol. 70, 3372-.377). ATI is one of the immunodominant antigens in mice, the immunodominant domains being located at the carboxy terminus of the molecule (Amegadzie et al. (1992), Virology 186, 777-782). The three clones found here, having identical reaction strengths, cover the range between bp 308 und 1437 and are therefore factually located C-terminally to centrally in the coded protein.

Plaque Size/host Range (ps/hr) Protein

The 38 or 45 kDa plaque size/host range protein (ps/hr) is coded by the ORF B5R (Takahashi-Nishimaki et al. (1991), Virology 181, 158-164). Ps/hr is a type 1 transmembrane protein which is incorporated into the membrane of extracellular virus particles or can be secreted by cells during the infection. Antibodies against ps/hr neutralize the infectiousness of the vaccinia virus (Galmiche et al. (1999), Virology 254, 71-80). Deletion of ps/hr causes an attenuation of the virus in vivo (Stern et al. (1997), Virology 233, 118-129). In addition, immunizing with B5R provides protection against an infection with otherwise lethal doses of the virus (Galmiche et al. (1999), Virology 254, 71-80). Three of the clones identified here are fragments which, in turn, cover the same region of this antigen and include the C terminus (FIG. 4C). This means that the sero-epitope represented by these clones is located in the extracellular area of this viral surface molecule and is thus easily accessible for antibodies.

### Example 5

Sequencing and Bioinformatic Analysis of the Identified Vaccinia Virus Antigens

Sequencing of the identified clones occurred according to standard techniques with oligonucleotides flanking the insert (BK-reverse, BK-universe) in Sanger's chain termination method. The determined sequences were compared through blast analysis with known sequences in the gene bank. The localization of the vaccinia virus antigens in the genome (accession number M35027) and the standard nomenclature are indicated in Table 3. This analysis shows that antigens distributed over the entire vaccinia virus genome were identified with the method according to the invention. So far, for a large number of identified genes, it has not been known that the gene products have an effect as antigens. By using the method according to the invention, one can therefore identify known antigens, but also unknown ones. Another advantage of the method according to the invention ist that antigens can be identified which are found on both strands (coding and complementary strand) of the genome.

TABLE 3
Identity, genomic localization, serological reactivity
and number of identified vaccinia virus antigens using
the method according to the invention.

| Vaccinia virus | Localization in | SEQ | | # |
|---|---|---|---|---|
| antigens | the VV genome | ID NO: | Signal | Clone |
| 39 kDa immunodominant | ORF 151 | 4 | ++++ | 62 |
| antigenic antigen (A4L) | (117270-116425) | | | |
| 94 kDa A-type inclusion | ORF 174 | 5 | +++ | 30 |
| protein (TA31L) | (138014-135837) | | | |
| 35 kDa plaque size/host | ORF 232 | 6 | +++ | 7 |
| range protein (B5R) | (167383-168336) | | | |
| 116 kDa DNA | ORF 80 | 7 | +++ | 4 |
| polymerase (E9L) | (59787-56767) | | | |
| 65 kDa envelope | ORF 60 | 8 | +++ | 2 |
| protein (F12L) | (43919-42012) | | | |
| 62 kDa rifampicin | ORF 145 | 9 | +++ | 1 |
| resistence gene (D13L) | (113026-111371) | | | |
| 32 kDa carbonic | ORF 137 | 10 | +++ | 1 |
| anhydrase-like protein | (107120-106206) | | | |
| (D8L) | | | | |
| 36 kDa late protein (I1L) | ORF 87 | 11 | +++ | 1 |
| | (63935-62997) | | | |
| 16 kDa protein (TC14L) | ORF 10 | 12 | +++ | 1 |
| | (10995-10567) | | | |
| 38 kDa serine protease | ORF 421 | 13 | ++ | 4 |
| inhibitor 2 (B13R) | (172562-172912 | | | |
| 18 kDa protein (C7L) | ORF 24 | 14 | ++ | 3 |
| | (19257-18805) | | | |
| 24.6 kDa protein (B2R) | ORF 226 | 15 | ++ | 2 |
| | (163876-164535) | | | |
| 36 kDa protein (A11R) | ORF 164 | 16 | ++ | 1 |
| | (124976-125932) | | | |
| 15 kDa membrane | ORF 167 | 17 | ++ | 1 |
| phosphoprotein (A14L) | (126785-127128) | | | |
| 147 kDa protein (J6R) | ORF 117 | 18 | ++ | 1 |
| | (86510-90370) | | | |
| 77 kDa protein (O1L)) | ORF 84 | 19 | ++ | 1 |
| | (62477-60477) | | | |
| 59 kDa protein (C2L) | ORF 30 | 20 | + | 4 |
| | (24156-22618) | | | |
| 90 kDa protein (D5R) | ORF 132 | 21 | + | 1 |
| | (101420-103777) | | | |
| 23 kDa protein (A17L) | ORF 170 | 22 | + | 1 |
| | (129314-128703) | | | |

FIG. 5 is a graphic representation of the vaccinia virus genome representing the open reading frame (ORF), showing that the antigens identified with the method according to the invention are distributed over the entire vaccinia virus genome. This indicates that the method according to the invention allows representative amplification of a specific pathogen nucleic acid from minimal amounts of source material (1-20 pg).

### Example 6

Representation Analysis Through Reverse Southern Blot

Representative amplification from minimal numbers of pathogen nucleic acids with the method according to the invention was also shown in the following experiment.

Ten gene segments of the vaccinia virus genome were selected and amplified through PCR reactions. Following separation by gel electrophoresis, the DNA fragments are blotted onto nylon membranes via alkaline transfer. Radioactive hybridization was performed using 20 ng of DNA produced according to the method according to the invention and marked ³²P. FIG. 6A shows that only a portion of the ten randomly selected segments of the genome are contained in a single Re-PCR DNA produced according to the method according to the invention. If several Re-PCR DNA produced in different batches and under varying conditions are combined, 100% representation of the randomly selected gene segments in the DNA produced according to the method according to the invention is evident (FIG. 6B). The varying abundance of the nucleic acids, i.e. of the 39 kDa antigen (FIG. 6B, Lane 10), can explain, at least in part, that certain gene segments are to be found more frequently in the DNA produced according to the method according to the invention. However, a low abundance of the DNA in the amplificate does not rule out frequent detection in screening, as is shown in the example of A-type inclusion protein DNA (FIG. 6B, Lane 2).

### Example 7

Differential Serology

Lambda phages whose recombinant inserts coded for antigens which were recognized by antibodies in the serum of infected mice, were tested for reactivity using serums from non-infected animals (immunologically naive) and serums from mice infected with a lymphocytary choriomeningitis virus. These studies were also performed as a plaque lift assay. It is thus easy to determine the antibody titer and the specific reactivity of the serums against the cloned antigens. In FIG. 7A it is shown how the reactivity of a serum obtained on day 21 after infection with the vaccinia virus directed against the 39 kDa antigen was determined. Double serum dilutions were incubated with recombinant 39 kDa antigen induced by phages in E. coli. Specific reactivity is still detectable at a serum dilution of 1:16,000. The time curve of the antibody reactivity against the 39 kDa antigen in mice infected with the vaccinia virus, lymphocytary choriomeningitis virus, and in non-infected mice is shown in FIG. 7B. The curve of the antibody response following infection with the vaccinia virus is typical for this infection. The absence of any reactivity against the 39 kDa antigen in naive mice and the only minor cross reactivity following infection with the lymphocytary choriomeningitis virus demonstrates the high diagnostic quality which can be achieved with antigens identified using the method according to the invention.

### Example 8

Identification of Bacterial Antigens by Means of the Method According to the Invention

First the conditions were elaborated that allow enrichment of bacterial pathogens from infected samples. For this, use was made of the fact that bacterial walls are resistant to lysis with solvents such as SDS. The stability of gram-negative, gram-positive bacteria and eukaryotic cells was determined by means of an SDS concentration series. As shown in FIG. 8, the corpuscular structure of gram-positive bacteria is maintained under 1% of SDS. Gram-negative bacteria as in this sample E. Coli do show resistance to lysis down to 0.1% of SDS. On the contrary, all membrane structures (cytoplasma, nucleus) of eukaryotic cells, such as fibroblasts in the present case, are completely lysed. The high SDS sensitivity of eukaryotic cells was also verified in other examples for leukocytes, spleen cells and lymph node biopsies. Following elaboration of the conditions, the method according to the invention was used for a hitherto insufficiently characterized pathogen, Tropheryma whippelii. Tropheryma whippellii is a gram-positive bacterium; infection with this pathogen can trigger Whipple's disease. Whipple's disease is a chronic infection of different organs, its principal manifestation being in the intestine, which can cause death without being diagnosed. This pathogen can be cultured in vitro only with difficulty, so that only minimal amounts of specific nucleic acid are available for molecular analyses. Because of these problems, analyzing the antigen structures of this pathogen has not been possible so far.

Using the method according to the invention, it was possible to define potential antigens of this pathogen. The essential steps leading to the characterization of Tropheryma whippelii-specific antigens are shown in FIG. 9.

Homogenized brain material containing T. whippelii coming from a patient who died of Whipple's disease was used to inoculate macrophages deactivated with interleukin-10 and Interleukin-4 (Schoedon et al. (1997) J Infect Dis. 176:672-677). Infected macrophages were harvested on day 7 following inoculation and 25 μL of the macrophage/bacteria mixture were processed. The differentiated cell lysis occurred through incubation of the bacteria-infected macrophages for 15 min at 55° C. in a proteinase K buffer containing 1% of SDS with 20 μg/mL of proteinase K. With this treatment the macrophages (eukaryotic cells) contained in the mixture were lysed. By lysing the macrophages, their nucleic acids (RNA, DNA) are realeased into the solution. By contrast, because the integrity of the gram-positive bacterial wall is maintained, the nucleic acids of the bacteria remain within the bacterial cells. Following incubation with SDS/proteinase K, the bacteria were pelleted by centrifuging the suspension. However, when no proteinase K was added, because of the high viscosity of the solution, the bacteria did not pellet as easily. The supernatant containing the nucleic acids of the macrophages was discarded and the hardly visible pellet washed repeatedly. Following the wash steps, pelleted bacteria were re-suspended in 100 μL of water, and amounts of 10 μL each of the suspension were used for light microscopy and, following dyeing with DAPI DNA dye in the immune fluorescence, for determining the bacteria count. According to the microscopic count, the number of bacteria isolated from 25 μL of the infected macrophages was approx. 4,000-6,000 DNA-containing particles. The residual 80 μL of enriched bacteria were subsequently used to obtain bacterial nucleic acids through standard techniques (cooking, denaturation, DNA isolation with phenol/chloroform). It was not possible to quantify experimentally the amount of DNA isolated from the bacteria because of the small quantity (no detectable signal in the EtBr gel). Because of the bacteria count determined through light and immune fluorescence microscopy (max. 6,000), a maximum yield of 6-12 pg of pathogen DNA was calculated for a hypothetical bacterial genome size of 1-2 million bases of double-stranded DNA (an average weight of the nucleotide of 660 was used in the calculation). 50% of the extracted DNA (i.e max. 3-6 pg) was amplified as described in the method according to the invention (Klenow, sequential PCR, Re-PCR), and a genomic library was established using the amplified fragments in lambda ZAP Express vector (Stratagene). Immunoscreening was performed with sera from patients infected with T. whippelii. Positive clones were sequenced and bioinformatically analyzed. As an example, FIG. 8 shows a clone that codes both for a bacterial putative lipoprotein and a putative histidine triad protein.

This example shows that, in addition to identifying viral antigens, the method according to the invention is also suited for identifying bacterial antigens.

### Example 9

Enrichment of Whipple's Bacteria from an Infected Spleen Sample from a Patient with Systemic Infection.

Samples of 20 μL each of cryopreserved spleen tissue from a patient with Morbus Whipple were used under five slightly modified conditions for enriching bacteria (a total of 100 μL). For this, the spleen samples were incubated in 1.5 mL of proteinase K buffer for 10-60 min at 55° C. with 20 mg/mL proteinase K added as described in Example 8. The bacteria in the infected spleen sample were then enriched by centrifugation as described in the above example and microscopically documented as described above. FIG. 10 shows a bacteria-rich pellet fraction. The bacteria were then digested by cooking in a GITC buffer and ultrasound treatment, and the bacterial nucleic acids were isolated in standard procedures. As expected, the amount of nucleic acids that were isolated from the enriched fractions was below the detection threshold of 1 ng. For documenting the bacterial enrichment, 1/100 each of the isolated nucleic acids was used for PCR amplification with Whipple's bacteria (sequence to be inserted) or human DNA-specific oligonucleotides (sequence to be inserted) and amplified through 37 cycles. FIG. 11A shows the results of the amplification (A: PCR-specific for Whipple's bacteria, B: PCR-specific for human DNA). The results are shown for amplification bands of the bacteria-enriched fractions (Lane 1-3) or the non-enriched fractions (Lane 4-6). Whereas, as expected, the amplification signals for human DNA in the non-enriched fractions are clearly less strong (Lane 4-6), fractions 1 and 2, in particular, display almost exclusively an amplification of pathogen nucleic acids. The example shows that the small amount of the material required makes it possible to vary the enrichment conditions slightly and then continue the procedure according to the invention with the most enriched pathogen fraction (in this case, 1 and 2).

### Example 10

Global amplification of pathogen nucleic acids directly from DNA isolated from a spleen sample. The pathogen nucleic acids isolated according to the method according to the invention, as shown in Example 9, were amplified as described in Example 3; a genomic library was then established in lambda ZAP Express vector (Stratagene) from the amplified fragments. Immunoscreening was performed with serums from patients infected with T. Whippelii. Positive clones were sequenced and bioinformatically analyzed.

1. Method for identifying biologically active structures encoded by the genome of microbial pathogens, using genomic pathogen nucleic acids, comprising the steps of:
(a) Extraction of genomic pathogen nucleic acids from pathogen-containing samples,
(b) Sequence-independent amplification of genomic pathogen nucleic acids,
(c) Expression of amplified pathogen nucleic acids and
(d) Screening and identification of biologically active structures.

2. Method according to claim 1, characterized in that the biologically active structures encoded by the genome of microbial pathogens are encoded by the genome of a bacterial pathogen.

3. Method according to claim 1, characterized in that the biologically active structures encoded by the genome of microbial pathogens are encoded by the genome of a DNA-containing virus.

4. Method according to claims 1-3, wherein the microbial pathogen is an intracellular viral or bacterial pathogen.

5. Method according to claims 1-3, wherein the microbial pathogen is an extracellular viral or bacterial pathogen.

6. Method according to claims 1-3, wherein the microbial pathogen is non-vital and/or non-infectious.

7. Method according to claim 1, wherein the biologically active structure is a pathogen antigen.

8. Method according to claim 1, wherein the biologically active structure is a pathogenicity factor of the microbial pathogen.

9. Method according to claim 1, wherein the biologically active structure is an enzymatically active protein.

10. Method according to claim 1, wherein 10-20 pg of pathogen nucleic acid are used for identification.

11. Method according to claim 1, wherein 1-10 pg of pathogen nucleic acid are used for identification.

12. Method according to claim 1, wherein the samples include blood, tissue, cultured cells, serum, secretions from lesions, and other body fluids.

13. Method according to claim 1, wherein the extraction of genomic pathogen nucleic acids from pathogen-containing samples in Step (a) comprises the following steps:
(a₁) Release of pathogen particles from pathogen-containing samples
(a₂) optional elimination and/or reduction of contaminating host nucleic acids and (a₃) extraction of genomic pathogen nucleic acid from released pathogen particles.

14. Method according to claim 13, wherein the release of pathogen particles in Step (a₁) occurs through cell lysis, sedimentation, centrifugation and/or filtration.

15. Method according to claim 13, wherein the elimination and/or reduction of contaminating host nucleic acids in Step (a₂) occurs through RNase- and/or DNase-digestion.

16. Method according to claim 13, wherein the extraction of the genomic pathogen nucleic acid in Step (a₃) occurs through separation of the genetic material of the pathogen from corpuscular components of the pathogen by proteinase K digestion, denaturation, lysozyme treatment or organic extraction.

17. Method according to claim 1, wherein the sequence-independent amplification of the genomic pathogen nucleic acid in Step (b) occurs through Klenow's reaction with adaptor oligonucleotides with degenerated 3′ end and subsequent PCR with oligonucleotides corresponding to the adaptor sequence.

18. Method according to claim 1, wherein amplification of the pathogen nucleic acid in Step (b) occurs by reverse transcription with degenerated oligonucleotides and subsequent PCR amplification.

19. Method according to claim 1, wherein amplification of the pathogen nucleic acid in Step (b) occurs through reverse transcription with degenerated oligonucleotides and subsequent amplification with T7 RNA polymerase.

20. Method according to claim 1, wherein, for expressing amplified pathogen nucleic acids in Step (c), introduction of pathogen nucleic acids into vectors and vector packaging in lambda phages occurs.

21. Method according to claim 1, wherein, for expressing amplified pathogen nucleic acids in Step (c), pathogen nucleic acids are introduced into filamentous phage vectors.

22. A method according to claims 20 and 21, wherein the vectors are selected from the group of viral, eukaryotic or prokaryotic vectors.

23. Method according to claim 1, wherein the screening is an immunoscreening for pathogen antigens, and identifying pathogen antigens in Step (d) comprises the following steps:
(d₁) infecting bacteria with lambda phages,
(d₂) culturing the infected bacteria by forming phage plaques,
(d₃) transferring phage plaques onto a nitrocellulose membrane or another solid phase suitable for immobilizing recombinant proteins derived from pathogens,
(d₄) incubating the membrane with serum or antibody-containing body fluids of the infected host,
(d₅) washing the membrane,
(d₆) incubating the membrane with a secondary AP-coupled anti-IgG-antibody which is specific for immunoglobulins of the infected host,
(d₇) detecting the clones reacting with host serum by colour reaction, and
(d₈) isolating and sequencing the reactive clones.

24. Method according to claim 1, wherein the screening is an immunoscreening for pathogen antigens and wherein identifying pathogen antigens in Step (d) comprises the following steps:
(d₁) generating recombinant filamentous phages by introducing the filamentous phage vectors into bacteria,
(d₂) incubating generated recombinant filamentous phages with serum from an infected host,
(d₃) selecting filamentous phages to which host immunoglobulins have bound, using immobilized reagents specific for the immunoglobulins of the infected host, and
(d₄) isolating and sequencing the selected clones.

25. Method according to claim 1, wherein the microbial pathogen, prior to the extraction of nucleic acids, is enriched by precipitation with polyethylene glycol, ultracentrifugation, gradient centrifugation or affinity chromatography.

26. Vaccinia virus antigen, characterized in that the antigen is encoded by a nucleic acid that is 80% homologous to one of the sequences SEQ ID NOS: 4,5,6,7,8,9,10, 11,12,13,14,15,16,17,18,19,20,21 or22.

27. Vaccinia virus antigen according to claim 26, characterized in that the antigen is encoded by a nucleic acid that is 90% homologous to one of the sequences SEQ ID NOS: 4,5,6,7,8,9,10,11,12,13,14,15,16,17,18,19,20,21 or22.

28. Vaccinia virus antigen according to claim 26, characterized in that the antigen is encoded by a nucleic acid that is 95% homologous to one of the sequences SEQ ID NOS: 4,5,6,7,8,9,10,11,12,13,14,15,16,17,18,19,20,21 or 22.

29. Vaccinia virus antigen according to claim 26, characterized in that the antigen is encoded by one of the nucleic acids SEQ ID NOS: 4,5,6,7,8,9,10,11,12,13,14, 15,16,17,18,19,20,21 oder 22.


SEQUENCE LISTING (22 sequences)

SEQ ID NO: 1 - DNA, 32 bp, Artificial
  Adaptor-N(6)-Oligonucleotide
gatgtaatacgaattggactcatatannnnnn

SEQ ID NO: 2 - DNA, 25 bp, Artificial
  EcoRI-Adaptor-Oligonucleotide
gatgtaatacgaattcgactcatat

SEQ ID NO: 3 - DNA, 24 bp, Artificial
  Mfe1-Adaptor-Oligonucleotide
gatgtaatacaattggactcatat

SEQ ID NO: 4 - DNA, 846 bp, Vaccinia virus
ttacttttggaatcgttcaaaacctttgactagttgtagaatttgatctattgccctacgcgtatactcccttgcatcatatacgttcgtcaccagatcgtttgtttcggcctgaagttggtgcatatctttttcaacactcgacatgagatccttaagggccatatcgtctagattttgttgagatgctgctcctggatttggattttgttgtgctgttgtacatactgtaccaccagtaggtgtaggagtacatacagtggccacaataggaggttgaggaggtgtaaccgttggagtagtacaagaaatatttccatccgattgttgtgtacatgtagttgttggtaacgtctgagaaggttgggtagatggcggcgtcgtcgttttttgatctttattaaatttagagataatatcctgaacagcattgctcggcgtcaacgctggaaggagtgaactcgccggcgcatcagtatcttcagacagccaatcaaaaagattagacatatcagatgatgtattagtttgttgtcgtggttttggtgtaggagcagtactactaggtagaagaataggagccggtgtagctgttggaaccggctgtggagttatatgaatagttggttgtagcggttggataggctgtctgctggcggccatcatattatctctagctagttgttctcgcaactgtctttgataatacgactcttgagactttagtcctatttcaatcgcttcatcctttttcgtatccggatccttttcttcagaataatagattgacgactttggtgtagaggattctgccagcccctgtgagaacttgttaaagaagtccat

SEQ ID NO: 5 - DNA, 2178 bp, Vaccinia virus
ctaagacgtcgcatctctctctgtttcggcattggtttcattattacgtctacagtcgttcaactgtctttcaagatctgatattctagattggagtctgctaatctctgtagcattttcacggcattcactcagttgtctttcaagatctgagattttagattggagtctgctaatctctgtaagatttcctcctccgctctcgatgcagtcggtcaacttattctctagttctctaatacgcgaacgcagtgcatcaacttcttgcgtgtcttcctggttgcgtgtacattcatcgagtctagattcgagatctctaacgcgtcgtcgttcttcctcaagttctctgcgtactacagaaagcgtgtccctatcttgttgatatttagcaatttctgattctagagtactgattttgcttacgtagttactaatatttgtcttggccttatcaagatcctccttgtatttgtcgcattccttgatatccctacgaagtctggacagttcccattcgacattacgacgtttatcgatttcagctcggagatcgtcatcgcgttgttttagccacatacgactgagttcaagttctcgttgacaagatccatctacttttccattcctaatagtatccagttccttttctagttctgaacgcatttctcgttccctatcaagcgattctctcaattctcggatagtcttcttatcaatttctaataaatctgaaccatcatctgtcccattttgaatatccctgtgttctttgatctcttttgtaagtcggtcgattctttcggttttataaacagaatccctttccaaagtcctaatcttactgagtttatcactaagttctgcattcaattcggtgagttttctcttggcttcttccaactctgttttaaactctccactatttccgcattcttcctcgcatttatctaaccattcaattagtttattaataactagttggtaatcagcgattcctatagccgttcttgtaattgtgggaacataattaggatcttctaatggattgtatggcttgatagcatcatctttatcattattagggggatggacaaccttaattggttggtcctcatctcctccagtagcgtgtggttcttcaataccagtgttagtaataggcttaggcaaatgcttgtcgtacgcgggcacttcctcatccatcaagtatttataatcgggttctacttcagaatattcttttctaagagacgcgacttcgggagttagtagaagaactctgtttctgtatctatcaacgctggaatcaatactcaagttaaggatagcgaatacctcatcgtcatcatccgtatcttctgaaacaccatcatatgacatttcatgaagtctaacgtattgataaatagaatcagatttagtattaaacagatccttaacctttttagtaaacgcatatgtatattttagatctccagatttcataatatgatcacatgccttaaatgtcagtgcttccatgatataatctggaacactaatgggtgatgaaaaagataccggaccatatgctacgttgataaataactctgaaccactaagtagataatgattaatgttaaggaagaggaaatattcagtatataggtatgtcttggcgtcatatcttgtactaaacacgctaaacagtttgttaatgtgatcaatttccaatagattaattagagcagcaggaataccaacaaacatattaccacatccgtattttctatgaatatcacatatcatgttaaaaaatcttgatagaagagcgaatatctcgtctgacttaatgagtcgtagttcagcagcaacataagtcataactgtaaatagaacatactttcctgtagtattgattctagactccgcatcaacaccattattaaaaatagttttatatacatctttaatctgctctccgttaatcgtcgaacgttctagtatacggaaacactttgatttcttatctgtagttaatgacttagtgatatcacgaagaatattacgaattacatttcttgtttttcttgagagacatgattcagaactcaactcatcgttccatagtttttctacctcagtggcgaaatctttggagtgcttggtacatttttcaataaggttcgtgacctccat

SEQ ID NO: 6 - DNA, 954 bp, Vaccinia virus
atgaaaacgatttccgttgttacgttgttatgcgtactacctgctgttgtttattcaacatgtactgtacccactatgaataacgctaaattaacgtctaccgaaacatcgtttaataataaccagaaagttacgtttacatgtgatcagggatatcattcttcggatccaaatgctgtctgcgaaacagataaatggaaatacgaaaatccatgcaaaaaaatgtgcacagtttctgattacatctctgaactatataataaaccgctatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatatgactatcaactgtgatgttggatatgaggttattggtgcttcgtacataagttgtacagctaattcttggaatgttattccatcatgtcaacaaaaatgtgatataccgtctctatctaatggattaatttccggatctacattttctatcggtggcgttatacatcttagttgtaaaagtggttttatactaacgggatctccatcatccacatgtatcgacggtaaatggaatcccgtactcccaatatgtgtacgaactaacgaagaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccgtaa

SEQ ID NO: 7 - DNA, 3021 bp, Vaccinia virus
ttatgcttcgtaaaatgtaggttttgaaccaaacattctttcaaagaatgagatgcataaaactttattatccaatagattgactatttcggacgtcaatcgtttaaagtaaacttcgtaaaatattctttgatcactgccgagtttaaaacttctatcgataattgtctcatatgttttaatatttacaagttttttggtccatggtacattagccggacaaatatatgcaaaataatatcgttctccaagttctatagtttctggattatttttattatattcagtaaccaaatacatattagggttatctgcggatttataatttgagtgatgcattcgactcaacataaataattctagaggagacgatctactatcaaattcggatcgtaaatctgtttctaaagaacggagaatatctatacatacctgattagaattcatccgtccttcagacaacatctcagacagtctggtcttgtatgtcttaatcatattcttatgaaacttggaaacatctcttctagtttcactagtacctttattaattctctcaggtacagattttgaattcgacgatgctgagtatttcatcgttgtatatttcttcttcgattgcataatcagattcttatataccgcctcaaactctattttaaaattattaaacaatactctattattaatcagtcgttctaactctttcgctatttctatagacttatcgacatcttgactgtctatctctgtaaacacggagtcggtatctccatacacgctacgaaaacgaaatctgtaatctataggcaacgatgttttcacaatcggattaatatctctatcgtccatataaaatggattacttaatggattggcaaaccgtaacataccgttagataactctgctccatttagtaccgattctagatacaagatcattctacgtcctatggatgtgcaactcttagccgaagcgtatgagtatagagcactatttctaaatcccatcagaccatatactgagttggctactatcttgtacgtatattgcatggaatcatagatggccttttcagttgaactggtagcctgttttaacatctttttatatctggctctctctgccaaaaatgttcttaatagtctaggaatggttccttctatcgatctatcgaaaattgctatttcagagatgaggttcggtagtctaggttcacaatgaaccgtaatatatctaggaggtggatatttctgaagcaatagctgattatttatttcttcttccaatctattggtactaacaacgacaccgactaatgtttccggagatagatttccaaagatacacacattaggatacagactgttataatcaaagattaatacattattactaaacattttttgttttggagcaaataccttaccgccttcataaggaaacttttgttttgtttctgatctaactaagatagttttagtttccaacaatagctttaacagtggacccttgatgactgtactcgctctatattcgaataccatggattgaggaagcacatatgttgacgcacccgcgtctgtttttgtttctactccataatactcccacaaatactgacacaaacaagcatcatgaatacagtatctagccatatctaaagctatgtttagattataatccttatacatctgagctaaatcaacgtcatcctttccgaaagataatttatatgtatcattaggtaaagtaggacataatagtacgactttaaatccattttcccaaatatctttacgaattactttacatataatatcctcatcaacagtcacataattacctgtggttaaaacctttgcaaatgcagcggctttgcctttcgcgtctgtagtatcgtcaccgataaacgtcatttctctaactcctctatttaatactttacccatgcaactgaacgcgttcttggatatagaatccaatttgtacgaatccaatttttcagatttttgaatgaatgaatatagatcgaaaaatatagttccattattgttattaacgtgaaacgtagtattggccatgccgcctactcccttatgactagactgatttctctcataaatacagagatgtacagcttcctttttgtccggagatctaaagataatcttctctcctgttaataactctagacgattagtaatatatctcagatcaaagttatgtccgttaaaggtaacgacgtagtcgaacgttagttccaacaattgtttagctattcgtaacaaaactatttcagaacatagaactagttctcgttcgtaatccatttccattagtgactgtatcctcaaacatcctctatcgacggcttcttgtatttcctgttccgttaacatctcttcattaatgagcgtaaacaataatcgtttaccacttaaatcgatataacagtaacttgtatgcgagattgggttaataaatacagaaggaaacttcttatcgaagtgacactctatatctagaaataagtacgatcttgggatatcgaatctaggtatttttttagcgaaacagttacgtggatcgtcacaatgataacatccattgttaatctttgtcaaatattgctcgtccaacgagtaacatccgtctggagatatcccgttagaaatataaaaccaactaatattgagaaattcatccatggtggcattttgtatgctgcgtttctttggctcttctatcaaccacatatctgcgacggagcattttctatctttaatatctagattataacttattgtctcgtcaatgtctatagttctcatctttcccaacggcctcgcattaaatggaggaggagacaatgactgatatatttcgtccgtcactacgtaataaaagtaatgaggaaatcgtataaatacggtctcaccatttcgacatctggatttcagatataaaaatctgttttcaccgtgactttcaaaccaattaatgcaccgaacatccat

SEQ ID NO: 8 - DNA, 1908 bp, Vaccinia virus
ttataattttaccatctgactcatggattcattaatatctttataagagctactaacgtataattctttataactgaactgagatatatacaccggatctatggtttccataattgagtaaatgaatgctcggcaataactaatggcaaatgtatagaacaacgaaattatactagagttgttaaagttaatattttctatgagctgttccaataaattatttgttgtgactgcgttcaagtcataaatcatcttgatactatccagtaaaccgtttttaagttctggaatattattatcccattgtaaagcccctaattcgactatcgaatatcctgctctgatagcagtttcaatatcgacggacgtcaatactgtaataaaggtggtagtattgtcatcatcgtgataaactactggaatatggtcgttagtaggtacggtaactttacacaacgcgatatataactttccttttgtaccatttttaacgtagttgggacgtcctgcagggtattgttttgaagaaatgatatcgagaacagatttgatacgatatttgttggattcctgattatttactataatataatctagacagatagatgattcgataaatagagaaggtatatcgttggtaggataatacatccccattccagtattctcggatactctattaatgacactagttaagaacatgtcttctattctagaaaacgaaaacatcctacatggactcattaaaacttctaacgctcctgattgtgtctcgaatgcctcgtacaaggatttcaaggatgccatagattctttgaccaacgatttagaattgcgtttagcatctgatttttttattaaatcgaatggtcggctctctggtttgctaccccaatgataacaatagtcttgtaaagataaaccgcaagaaaatttatacgcatccatccaaataaccctagcaccatcggatgatattaatgtattattatagattttccatccacaattattgggccagtatactgttagcaacggtatatcgaatagattactcatgtaacctactagaatgatagttcgtgtactagtcataatatctttaatccaatctaagaaatttaaaattagattttttacactgttaaagttaacaaaggtattacccgggtacgtggatatcatatatggtattggtccattatcagtaatagctccataaactgatacggcgatggtttttatatgtgtttgatctaacgaggaagaaattcgcacccacaattcatctctagatatgtatttaatatcaaacggtaacacatcaatttcgggacgcgtatatgtttctaaatttttaatccaaatataatgatgacctatatgccctattatcatactgtcaactatagtacacctagagaacttacgatacatctgtttcctataatcgttaaattttacaaatctataacatgctaaaccttttgacgacaaccattcattaatttctgatatggaatctgtattctcgataccgtattgttctaaagccagtgctatatctccctgttcgtgggaacgctttcgtataatatcgatcaacggataatctgaagtttttggagaataatatgactcatgatctatttcgtccataaacaatctagacataggaattggaggcgatgatcttaattttgtgcaatgagtcgtcaatcctataacttctaatcttgtaatattcatcatcgacataatactatctatgttatcatcgtatattagtataccatgaccttcttcatttcgtgccaaaatgatatacagtcttaaatagttacgcaatatctcaatagtttcataattgttagctgttttcatcaagatttgtaccctgtttaacat

SEQ ID NO: 9 - DNA, 1656 bp, Vaccinia virus
ttagttattatctcccataatcttggtaatacttaccccttgatcgtaagataccttatacaggtcattacatacaactaccaattgtttttgtacataatagattggatggttgacatccatggtggaataaactactcgaacagatagtttatctttccccctagatacattggccgtaatagttgtcggcctaaagaatatctttggtgtaaagttaaaagttagggttcttgttccattattgctttttgtcagtagttcattataaattctcgagatgggtccgttctctgaatatagaacatcatttccaaatctaacttctagtctagaaataatatcggtcttattcttaaaatctattcccttgatgaagggatcgttaatgaacaaatccttggcctttgattcggctgatctattatctccgttatagacgttacgttgactagtccaaagacttacaggaatagatgtatcgatgatgttgatactatgtgatatgtgagcaaagattgttctcttagtggcatcactatatgttccagtaatggcggaaaactttttagaaatgttatatataaaagaattttttcgtgttccaaacattagcagattagtatgaagataaacactcatattatcaggaacattatcaatttttacatacacatcagcatcttgaatagaaacgataccatcttctggaacctctacgatctcggcagactccggataaccagtcggtgggccatcactaacaataactagatcatccaacaatctactcacatatgcatctatataatctttttcatcttgtgagtaccctggatacgaaataaatttattatccgtatttccataataaggtttagtataaacagagagcgatgttgccgcatgaacttcagttacagtcgccgttggttggtttatttgacctattactctcctaggtttctctataaacgatggtttaatttgtacattcttaaccatatatccaataaagctcaattcaggaacataaacaaattctttgttgaacgtttcaaagtcgaacgaagagtcacgaataacgatatcggatactggattgaaggttaccgttacggtaatttttgaatcggatagtttaagactgctgaatgtatcttccacatcaaacggagttttaatataaacgtatactgtagatggttctttaatagtgtcattaggagttaggccaatagaaatatcattaagttcactagaatatccagagtgtttcaaagcaattgtattattgatacaattattatataattcttcgccctcaatttcccaaataacaccgttacacgaagagatagatacgtgattaatacatttatatccaacatatggtacgtaaccgaatcttcccatacctttaacttctggaagttccaaactcagaaccaaatgattaagcgcagtaatatactgatccctaatttcgaagctagcgatagcctgattgtctggaccatcgtttgtcataactccggatagagaaatatattgcggcatatataaagttggaatttgactatcgactgcgaagacattagaccgtttaatagagtcatccccaccgatcaaagaattaatgatagtattattcat

SEQ ID NO: 10 - DNA, 915 bp, Vaccinia virus
ctagttttgtttttctcgcgaatatcgtcgactcataagaaagagaatagcggtaagtataaacacgaatactatggcaataattgcgaatgttttattcccttcgatatatttttgataatatgaaaaacatgtctctctcaaatcggacaaccatctcataaaatagttctcgcgcgctggagaggtagttgctgctcgtataatctccccagaataatatacttgcgtgtcgtcgttcaatttatacggatttctatagttctctgttatataatacggttttccatcatgattagacgacgacaatagtgttctaaatttagatagttgatcagaatgaatgtttattggcgttggaaaaattatccatacagcgtctgcagagtgcttgatagttgttcctagatatgtaaaataatccaacgtactaggtagcaaattgtctagataaaatactgaatcaaacggcgcagacgtattagcggatctaatggaatccaattgattgactatcttttgaaaatatacatttttatgatccgatacttgtaagaatatagaaataatgataagtccatcatcgtgtttttttgcctcttcataagaactatattttttcttattccaatgaacaagattaatctctccagagtatttgtacacatctatcaagtgattggatccataatcgtcttcctttccccaatatatatgtagtgatgataacacatattcattggggagaaaccctccacttatatatcctcctttaaaattaatccttactagttttccagtgttctggatagtggttggtttcgactcattataatgtatgtctaacggcttcaatcgcgcgttagaaattgcttttttagtttctatattaataggagatagttgttgcggcat

SEQ ID NO: 11 - DNA, 939 bp, Vaccinia virus
ttattcagcattacttgatatagtaatattaggcacagtcaaacattcaaccactctcgatacattaactctctcattttctttaacaaattctgcaatatcttcgtaaaaagattcttgaaactttttagaatatctatcgactctagatgaaatagcgttcgtcaacatactatgttttgtatacataaaggcgcccattttaacagtttctagtgacaaaatgctagcgatcctaggatcctttagaatcacatagattgacgattcgtctctcttagtaactctagtaaaataatcatacaatctagtacgcgaaataatattatccttgacttgaggagatctaaacaatctagttttgagaacatcgataagttcatcgggaatgacatacatactatctttaatagaactcttttcatccagttgaatggattcgtccttaaccaactgattaatgagatcttctattttatcattttccagatgatatgtatgtccattaaagttaaattgtgtagcgcttctttttagtctagcagccaatactttaacatcactaatatcgatatacaaaggagatgatttatctatggtattaagaattcgtttttcgacatccgtcaaaaccaattcctttttgcctgtatcatccagttttccatcctttgtaaagaaattattttctactagactattaataagactgataaggattcctccataattgcacaatccaaactttttcacaaaactagactttacgagatctacaggaatgcgtacttcaggtttcttagcttgtgattttttcttttgcggacattttcttgtgaccaactcatctaccatttcattgattttagcagtgaaataagctttcaatgcacgggcactgatactattgaaaacgagttgatcttcaaattccgccat

SEQ ID NO: 12 - DNA, 429 bp, Vaccinia virus
ttaattgcaaagatctatataatcattatagcgttgacttatggactctggaatcttagacgatgtacagtcatctataatcatggcatatttaatacattgttttatagcatagtagttatctacgatgttagatatttctctcaatgaatcaatcacataatctaatgtaggtttatgacataatagcattttcagcagttcaatgtttctagattcgttgatggcaatggctatacatgtatatccgttatttgatctaatgttgacatctgaaccggattctagcagtaaagatactagagattgtttattatatctaacagccttgtgaagaagtgtttctcctcgtttgtcaatcatgttaatgtctttaagataaggtaggcaaatgtttatagtactaagaattgggcaagcataagacat

SEQ ID NO: 13 - DNA, 1038 bp, Vaccinia virus
atggatatcttcagggaaatcgcatcttctatgaaaggagagaatgtattcatttctccagcgtcaatctcgtcagtattgacaatactgtattatggagctaatggatccactgctgaacagctatcaaaatatgtagaaaaggaggagaacatggataaggttagcgctcaaaatatctcattcaaatccataaataaagtatatgggcgatattctgccgtgtttaaagattcctttttgagaaaaattggcgataagtttcaaactgttgacttcactgattgtcgcactatagatgcaatcaacaagtgtgtagatatctttactgaggggaaaatcaatccactattggatgaaccattgtctcctgatacctgtctcctagcaattagtgccgtatactttaaagcaaaatggttgacgccattcgaaaaggaatttaccagtgattatcccttttacgtatctccgacggaaatggtagatgtaagtatgatgtctatgtacggcaaggcatttaatcacgcatctgtaaaggaatcattcggcaacttttcaatcatagaactgccatatgttggagatactagtatgatggtcattcttccagacaagattgatggattagaatccatagaacaaaatctaacagatacaaattttaagaaatggtgtaactctctggaagctacgtttatcgatgttcacattcccaagtttaaggtaacaggctcgtataatctggtggatactctagtaaagtcaggactgacagaggtgttcggttcaactggagattatagcaatatgtgtaattcagatgtgagtgtcgacgctatgatccacaaaacgtatatagatgtcaatgaagagtatacagaagcagctgcagcaacttgtgcactggtgtcagactgtgcatcaacaattacaaatgagttctgtgtagatcatccgttcatctatgtgattaggcatgttgatggaaaaattcttttcgttggtagatattgctctccgacaactaattgttaa

SEQ ID NO: 14 - DNA, 453 bp, Vaccinia virus
ttaatccatggactcataatctctatacgggattaacggatgttctatatacggggatgagtagttctcttctttaactttatactttttactaatcatatttagactgatgtatgggtaatagtgtttgaagagctcgttctcatcatcagaataaatcaatatctctgtttttttgttatacagatgtattacagcctcatatattacgtaatagaacgtgtcatctaccttattaactttcaccgcatagttgtttgcaaatacggttaatcctttgacctcgtcgatttccgaccaatctgggcgtataatgaatctaaactttaatttcttgtaatcattcgaaataatttttagtttgcatccgtagttatcccctttatgtaactgtaaatttctcaacgcgatatctccattaataatgatgtcgaattcgtgctgtatacccat

SEQ ID NO: 15 - DNA, 660 bp, Vaccinia virus
atggcgatgttttacgcacacgctctcggtgggtacgacgagaatcttcatgcctttcctggaatatcatcgactgttgccaatgatgtcaggaaatattctgttgtgtcagtttataataacaagtatgacattgtaaaagacaaatatatgtggtgttacagtcaggtgaacaagagatatattggagcactgctgcctatgtttgagtgcaatgaatatctacaaattggagatccgatccatgatcaagaaggaaatcaaatctctatcatcacatatcgccacaaaaactactatgctctaagcggaatcgggtacgagagtctagacttgtgtttggaaggagtagggattcatcatcacgtacttgaaacaggaaacgctgtatatggaaaagttcaacatgattattctactatcaaagagaaggccaaagaaatgagtacacttagtccaggacctataattgattaccacgtctggataggagattgtatctgtcaagttactgctgtggacgtacatggaaaggaaattatgagaatgagattcaaaaagggtgcggtgcttccgatcccaaatctggtaaaagttaaacttggggagaatgatacagaaaatctttcttctactatatcggcggcaccatcgaggtaa

SEQ ID NO: 16 - DNA, 957 bp, Vaccinia virus
atgacgaccgtaccagtgacggatatacaaaacgatttaattacagagttttcagaagataattatccatctaacaaaaattatgaaataactcttcgtcaaatgtctattctaactcacgttaacaacgtggtagatagagaacataatgccgccgtagtgtcatctccagaggaaatatcctcacaacttaatgaagatctatttccagatgatgattctccggccactattatcgaaagagtacaacctcatactactattattgacgatactccacctcctacgtttcgtagagagttattgatatcggaacaacgtcaacaacgagaaaaaagatttaatattacagtatcgaaaaatgctgaagcaataatggaatctagatctatgatatcttctatgccaacacaaacaccatccttgggagtagtttatgataaagataaaagaattcagatgttggaggatgaagtggttaatcttagaaatcaacgatctaatacaaaatcatctgataatttagataattttaccagaatactatttggtaagactccgtataaatcaacagaagttaataagcgtatagccatcgttaattatgcaaatttgaacgggtctcccttatcagtcgaggacttggatgtttgttcagaggatgaaatagatagaatctataaaacgattaaacaatatcacgaaagtagaaaacgaaaaattatcgtcactaacgtgattattattgtcataaacattatcgagcaagcattgctaaaactcggatttgaagaaatcaaaggactgagtaccgatatcacttcagaaattatcgatgtggagatcggagatgactgcgatgctgtagcatctaaactaggaatcggtaacagtccggttcttaatattgtattgtttatactcaagatattcgttaaacgaattaaaattatttaa

SEQ ID NO: 17 - DNA, 273 bp, Vaccinia virus
ttagttcatggaaatatcgctatgattggtatgaatgactccgctaactctgtggggtgcgcagtgctttccccacatagaataaattagcattccgactgtgataataataccaagtataaacgccataatactcaatactttccatgtacgagtgggactggtagacttactaaagtcaataaaggcgaagatacacgaaagaatcaaaagaatgattccagcgattagcacgccggaaaaataatttccaatcataagcatcatgtccat

SEQ ID NO: 18 - DNA, 3861 bp, Vaccinia virus
atggctgtaatctctaaggttacgtatagtctatatgatcaaaaagagattaatgctacagatattatcattagtcatgttaaaaatgacgacgatatcggtaccgttaaagatggtagactaggtgctatggatggggcattatgtaagacttgtgggaaaacggaattggaatgtttcggtcactggggtaaagtaagtatttataaaactcatatagttaagcctgaatttatttcagaaattattcgtttactgaatcatatatgtattcactgcggattattgcgttcacgagaaccgtattccgacgatattaacctaaaagagttatcgggacacgctcttaggagattaaaggataaaatattatccaagaaaaagtcatgttggaacagcgaatgtatgcaaccgtatcaaaaaattactttttcaaagaaaaaggtttgtttcgtcaacaagttggatgatattaacgttcctaattctctcatctatcaaaagttaatttctattcatgaaaagttttggccattattagaaattcatcaatatccagctaacttattttatacagactactttcccatccctccgctgattattagaccggctattagtttttggatagatagtatacccaaagagaccaatgaattaacttacttattaggtatgatcgttaagaattgtaacttgaatgctgatgaacaggttatccagaaggcggtaatagaatacgatgatattaaaattatttctaataacacttccagtatcaatttatcatatattacatccggcaaaaataatatgattagaagttatattgtcgcccgacgaaaagatcagaccgctagatctgtaattggtcccagtacatctatcaccgttaatgaggtaggaatgcccgcatatattagaaatacacttacagaaaagatatttgttaatgcctttacagtggataaagttaaacaactattagcgtcaaaccaagttaaattttactttaataaacgattaaaccaattaacaagaatacgccaaggaaagtttattaaaaataaaatacatttattgcctggtgattgggtagaagtagctgttcaagaatatacaagtattatttttggaagacagccgtctctacatagatacaacgtcatcgcttcatctatcagagctaccgaaggagatactatcaaaatatctcccggaattgccaactctcaaaatgctgatttcgacggagatgaagaatggatgatattggagcaaaatcctaaagccgtaattgaacaaagtattcttatgtatccgacgacgttactcaaacacgatattcatggagcccccgtttatggatctattcaagatgaaatcgtagcagcgtattcattgtttagaatacaagatctttgtttagatgaagtattgaacatcttggggaaatatggaagaaagttcgatcctaaaggtaaatgtaaattcagcggtaaagatatctatacttacttgataggtgaaaagattaattatccgggtctcttaaaggatggtgaaattattgcaaacgacgtagatagtaattttgttgtggctatgaggcatctgtcattggctggactcttatccgatcataagtcgaacgtggaaggtatcaactttattatcaagtcatcttatgtttttaagagatatctatctatttacggttttggggtgacattcaaagatctgagaccaaattcgacgttcactaataaattggaggccatcaacgtagaaaaaatagaacttatcaaagaagcatacgccaaatatctcaacgatgtaagagacgggaaaatagttccattatctaaagctttagaggcggactatgtggaatccatgttatccaacttgacaaatcttaatatccgagagatagaagaacatatgagacaaacgctgatagatgatccagataataacctcctgaaaatggccaaagcgggttataaagtaaatcccacagaactaatgtatattctaggtacttatggacaacagaggattgatggtgaaccagcagagactcgagtattgggtagagtcttaccttactatcttccagactctaaggatccagaaggaagaggttatattcttaattctttaacaaaaggattaacaggttctcaatattacttttcgatgctggttgccagatctcaatctactgatatcgtctgtgaaacatcacgtaccggaacactggctagaaaaatcattaaaaagatggaggatatggtggtcgacggatacggacaagtagttataggtaatacgctcatcaagtacgccgccaattataccaaaattctaggctcagtatgtaaacctgtagatcttatctatccagatgagtccatgacttggtatttggaaattagtgctctgtggaataaaataaaacagggattcgtttactctcagaaacagaaacttgcaaaaaagacattggcgccgtttaatttcctagtattcgtcaaacccaccactgaggataatgctattaaggttaaggatctgtacgatatgattcataacgtcattgatgatgtgagagagaaatacttctttacggtatctaatatagattttatggagtatatattcttgacgcatcttaatccttctagaattagaattacaaaagaaacggctatcactatctttgaaaagttctatgaaaaactcaattatactctaggtggtggaactcctattggaattatttctgcacaggtattgtctgagaagtttacacaacaagccctgtccagttttcacactactgaaaaaagtggtgccgtcaaacaaaaacttggtttcaacgagtttaataacctgactaatttgagtaagaataagaccgaaattatcactctggtatccgatgatatctctaaacttcaatctgttaagattaatttcgaatttgtatgtttgggagaattaaatccaaacatcactcttcgaaaagaaacagataggtatgtagtagatataatagtcaatagattatacatcaagagagcagaaattaccgaattagtcgtcgaatatatgattgaacgatttatctcctttagcgtcattgtaaaggaatggggtatggagacattcattgaggacgaggataatattagatttactgtctacctaaatttcgttgaaccggaagaattgaatcttagtaagtttatgatggttcttccgggtgccgccaacaagggcaagattagtaaattcaagattcctatctctgactatacgggatatgacgacttcaatcaaacaaaaaagctcaataagatgactgtagaactcatgaatctaaaagaattgggttctttcgatttggaaaacgtcaacgtgtatcctggagtatggaatacatacgatatcttcggtatcgaggccgctcgtgaatacttgtgcgaagccatgttaaacacctatggagaagggttcgattatctgtatcagccttgtgatcttctcgctagtttactatgtgctagttacgaaccagaatcagtgaataaattcaagttcggcgcagctagtactcttaagagagctacgttcggagacaataaagcattgttaaacgcggctcttcataaaaagtcagaacctattaacgataatagtagctgccacttttttagcaaggtccctaatataggaactggatattacaaatactttatcgacttgggtcttctcatgagaatggaaaggaaactatctgataagatatcttctcaaaagatcaaggaaatggaagaaacagaagacttttaa

SEQ ID NO: 19 - DNA, 2001 bp, Vaccinia virus
ttaacgagttccatttatatcatccaatattattgaaatgacgttgatggacagatgatacaaataagaaggtacggtacctttgtccaccatctcctccaattcatgctctattttgtcattaactttaatgtatgaaaacagtacgccacatgcttccatgacagtgtgtaacactttggatacaaaatgtttgacattagtataattgtttaagactgtcaatctataatagatagtagctataatatattctatgatggtattgaagaagatgacaatcttggcatattgatcatttaacacagacatggtatcaacagatagcttgaatgaaagagaatcagtaattggaataagcgtcttctcgatggagtgtccgtataccaacatgtctgatattttgatgtattccattaaattatttagttttttctttttattctcgttaaacagcatttctgtcaacggaccccaacatcgttgaccgattaagttttgattgatttttccgtgtaaggcgtatctagtcagatcgtatagcctatccaataatccatcatctgtgcgtagatcacatcgtacactttttaattctctatagaagagcgacagacatctggaacaattacagacagcaatttctttattctctacagatgtaagatacttgaagacattcctatgatgatgcagaattttggataacacggtattgatggtatctgttaccataattcctttgatggctgatagtgtcagagcacaagatttccaatctttgacaatttttagcaccattatctttgttttgatatctatatcagacagcatggtgcgtctgacaacacagggattaagacggaaagatgaaatgattctctcaacatcttcaatggataccttgctattttttctggcattatctatatgtgcgagaatatcctctagagaatcagtatcctttttgatgatagtggatctcaatgacatgggacgtctaaaccttcttattctatcaccagattgcatggtgatttgtcttctttcttttatcataatgtaatctctaaattcatcggcaaattgtctatatctaaaatcataatatgagatgtttacctctacaaatatctgttcgtccaatgttagagtatctacatcagttttgtattccaaattaaacatggcaacggatttaattttatattcctctattaagtcctcgtcgataataacagaatgtagataatcatttaatccatcgtacatggttggaagatgcttgttgacaaaatctttaattgtcttgatgaaggtgggactatatctaacatcttgattaataaaatttataacattgtccataggatactttgtaactagttttatacacatctcttcatcggtaagtttagacagaatatcgtgaacaggtggtatattatattcatcagatatacgaagaacaatgtccaaatctatattgtttaatatattatatagatgtagtgtagctcctacaggaatatctttaactaagtcaatgatttcatcaaccgttagatctattttaaagttaatcatataggcattgatttttaaaaggtatgtagccttgactacattctcattaattaaccattccaagtcactgtgtgtaagaagattatattctatcataagcttgactacatttggtcccgataccattaaagaattcttatgatataaggaaacagcttttaggtactcatctactctacaagaattttggagagccttaacgatatcagtgacgtttattatttcaggaggaaaaaacctaacattgagaatgtcggagttaatagcttccagatacagtgattttggcaatagtccgtgtaatccataatccagtaacacgagctggtgcttgctagacaccttttcaatgtttaatttttttgaaataagctttgataaagccttcctcgcaaattccggatacatgaacat

SEQ ID NO: 20 - DNA, 1539 bp, Vaccinia virus
ctattgtagaaattgtttttcacagttgctcaaaaacgatggcagtgacttatgagttacgttacactttggagtctcatctttagtaaacatatcataatattcgatattacgagttgacatatcgaacaaattccaagtatttgattttggataatattcgtattttgcatctgctataattaagatataatcaccgcaagaacacacgaacatctttcctacatggttaaagtacatgtacaattctatccatttgtcttccttaactatatatttgtatagataattacgagtctcgtgagtaattccagtaattacatagatgtcgccgtcgtactctacagcataaactatactatgatgtctaggcatgggagacttttttatccaacgatttttagtgaaacattccacatcgtttaatactacatatttttcatacgtggtataaactccacccattacatatatatcatcgtttacgaataccgacgcgcctgaatatctaggagtaattaagtttggaagtcttatccatttcgaagtgccgtgtttcaaatattctgccacacccgttgaaatagaaaattctaatcctcctattacatataactttccatcgttaacacaagtactaacttctgattttaacgacgacatattagtaaccgttttccattttttcgtttcaagatctacccgcgatacggaataaacatgtctattgttaatcatgccgccaataatgtatagacaattatgtaaaacatttgcattatagaattgtctatctgtattaccgactatcgtccaatattctgtcctaggagagtaatgggttattgtggatatataatcagagtttttaatgactactatattatgttttataccatttcgtgtcactggctttgtagatttggatatagttaatcccaacaatgatatagcattgcgcatagtattagtcataaacttgggatgtaaaatgttgatgatatctacatcgtttggatttttatgtatccactttaataatatcatagctgtaacatcctcatgatttacgttaacgtcttcgtgggataagatagttgtcagttcatcctttgataattttccaaattctggatcggatgtcaccgcagtaatattgttgattatttctgacatcgacgcattatatagttttttaattccatatcttttagaaaagttaaacatccttatacaatttgtggaattaatattatgaatcatagtttttacacatagatctactacaggcggaacatcaattattacggcagcaactagtatcatttctacattgtttatggtgatgtttatcttcttccagcgcatatagtctaatagcgattcaaacgcgtgatagtttataccattcaatataatcgcttcatcctttagatggtgatcctgaatgcgtttaaaaaaattatacggagacgccgtaataatttccttattcacttgtataatttccccattgatagaaaatatcacgctttccat

SEQ ID NO: 21 - DNA, 2358 bp, Vaccinia virus
atggatgcggctattagaggtaatgatgttatctttgtccttaagactataggtgtcccatcagcatgtagacaaaatgaagatccaagattcgtagaagcatttaaatgcgacgagttaaaaagatatattgataataatccagaatgtacactattcgaaagtcttagggatgaggaagcatactctatagtcagaattttcatggatgtagatttagacgcgtgtctagacgaaatagattatttaacggctattcaagattttattatcgaggtgtcaaactgtgtagctagattcgcgtttacagaatgcggtgccattcatgaaaatgtaataaaatccatgagatctaatttttcattgactaagtctacaaatagagataaaacaagttttcatattatctttttagacacgtataccactatggatacattgatagctatgaaacgaacactattagaattaagtagatcatctgaaaatccactaacaagatcgatagacactgccgtatataggagaaaaacaactcttcgggttgtaggtactaggaaaaatccaaattgcgacactattcatgtaatgcaaccaccgcatgataatatagaagattacctattcacttacgtggatatgaacaacaatagttattacttttctctacaacgacgattggaggatttagttcctgataagttatgggaaccagggtttatttcattcgaagacgctataaaaagagtttcaaaaatattcattaattctataataaactttaatgatctcgatgaaaataattttacaacggtaccactggtcatagattacgtaacaccttgtgcattatgtaaaaaacgatcgcataaacatccgcatcaactatcgttggaaaatggtgctattagaatttacaaaactggtaatccacatagttgtaaagttaaaattgttccgttggatggtaataaactgtttaatattgcacaaagaattttagacactaactctgttttattaaccgaacgaggagactatatagtttggattaataattcatggaaatttaacagcgaagaacccttgataacaaaactaattctgtcaataagacatcaactacctaaggaatattcaagcgaattactctgtccgaggaaacgaaagactgtagaagctaacatacgagacatgttagtagattcagtagagaccgatacctatccggataaacttccgtttaaaaatggtgtattggacctggtagacggaatgttttactctggagatgatgctaaaaaatatacgtgtactgtatcaaccggatttaaatttgacgatacaaagttcgtcgaagacagtccagaaatggaagagttaatgaatatcattaacgatatccaaccattaacggatgaaaataagaaaaatagagagctatatgaaaaaacattatctagttgtttatgtggtgctaccaaaggatgtttaacattcttttttggagaaactgcaactggaaagtcgacaaccaaacgtttgttaaagtctgctatcggtgacctgtttgttgagacgggtcaaacaattttaacagatgtattggataaaggacctaatccatttatcgctaacatgcatttgaaaagatctgtattctgtagcgaactacctgattttgcctgtagtggatcaaagaaaattagatctgataatattaaaaagttgacagaaccttgtgtcattggaagaccgtgtttctccaataaaattaataatagaaaccatgctacaatcattatcgatactaattacaaacctgtctttgataggatagataacgcattaatgagaagaattgccgtcgtgcgattcagaacacacttttctcaaccttctggtagagaggctgctgaaaataatgacgcgtacgataaagtcaaactattagacgaggggttagatggtaaaatacaaaataatagatatagatttgcatttctatacttgttggtgaaatggtacaaaaaatatcatgttcctattatgaaactatatcctacacccgaagagattcctgactttgcattctatctcaaaataggtactctgttagtatctagctctgtaaagcatattccattaatgacggacctctccaaaaagggatatatattgtacgataatgtggttactcttccgttgactactttccaacagaaaatatccaagtattttaattctagactatttggacacgatatagagagcttcatcaatagacataagaaatttgccaatgttagtgatgaatatctgcaatatatattcatagaggatatttcatctccgtaa

SEQ ID NO: 22 - DNA, 612 bp, Vaccinia virus
ttaataatcgtcagtatttaaactgttaaatgttggtatatcaacatctaccttatttcccgcagtataaggtttgttgcaggtatactgttcaggaatggttacatttatacttcttctatagtcctgtctttcgatgttcatcacatatgcaaagaacagaataaacaaaataatgtaagaaataatattaaatatctgtgaattcgtaaatacattgattgccataataattacagcagctacaatacacacaatagacattcccacagtgttgccattacctccacgatacatttgagttactaagcaataggtaataactaagctagtaagaggcaatagaaaagatgagataaatatcatcaatatagagattagaggagggctatatagagccaagacgaacaaaatcaaaccgagtaacgttctaacatcattatttttgaagattcccaaataatcattcattcctccataatcgttttgcatcatacctccatctttaggcataaacgattgctgctgttcctctgtaaataaatctttatcaagcactccagcacccgcagagaagtcgtcaagcatattgtaatatcttaaataactcat